(19) United States
(12) Patent Application Publication
(10) Pub. No.: US 2025/0041440 A1
(43) Pub. Date: Feb. 6, 2025

(54) COMPOSITIONS AND METHODS FOR PREVENTION OR REDUCTION OF CARPA

(71) Applicant: The Trustees of the University of Pennsylvania, Philadelphia, PA (US)

(72) Inventors: Jacob Brenner, Princeton Junction, NJ (US); Jacob W. Myerson, Clayton, MO (US); Vladimir R. Muzykantov, Bryn Athyn, PA (US); Zhicheng Wang, Wayne, PA (US); Elizabeth D. Hood, Philadelphia, PA (US); Jia Nong, Wayne, PA (US); Wenchao Song, Bryn Mawr, PA (US)

(73) Assignee: The Trustees of the University of Pennsylvania, Philadelphia, PA (US)

(21) Appl. No.: 18/717,294

(22) PCT Filed: Dec. 7, 2022
(86) PCT No.: PCT/US2022/081102

§ 371 Date: Jun. 6, 2024

Related U.S. Application Data
(60) Provisional application No. 63/286,938, filed on Dec. 7, 2021.

(51) Int. Cl. A61K 47/69; A61K 9/51; A61K 38/00; C12N 15/113

(52) U.S. Cl. CPC A61K 47/6913; A61K 9/5123; C12N 15/113; A61K 38/00; C12N 2310/16

(57) ABSTRACT

Provided herein are compositions and methods for preventing or reducing CARPA in a subject in need thereof. In one aspect, the composition comprises a therapeutic molecule, diagnostic molecule, molecular complex, or nanoparticle having a complement inhibitory protein attached thereto.

This invention was made with government support under HL153510, HL143806, HL155106, HL157189, AI146162, and AI148797, awarded by the National Institutes of Health. The government has certain rights in the invention.

### BACKGROUND OF THE INVENTION

Nanomedicine has long sought an arsenal of nanoparticles that, upon intravascular injection, display a prolonged circulation time, the ability to target specific cells and organs, and minimal side effects from the nanoparticle itself. Unfortunately, each of these goals has been continually impeded by one of the oldest parts of the immune system, the complement protein cascade^{[1-3]}. Complement proteins comprise ˜40 proteins in the blood that evolved over 500 million years to rapidly opsonize (bind to the surface of) microbes in order to mobilize the immune system to clear the pathogen^{[4]}. Given that engineered nanoparticles share with microbes a similar size scale and many similarities in surface chemistry, it is not surprising that complement similarly attacks therapeutic nanoparticles, causing increased phagocytosis by the reticulo-endothelial system (RES), decreased circulation time, fouling of targeting antibodies, and anaphylactoid side effects. Thus, engineering new materials to evade complement has been among nanomedicine's longest and most-sought-after goals^{[5]}.

The focal point of the complement system is the protein C3, whose highly reactive thioester forms covalent bonds to nucleophiles on non-self surfaces such as microbes and nanoparticles (FIG. 1a)^{[6]}. This reaction leaves the surface covalently bound to a large protein fragment, C3b, which initiates rapid interaction with other complement proteins to form an enzymatic complex (C3bBb) which catalyzes other C3 molecules to bond to nearby surface nucleophiles. This positive feedback loop, called the amplification loop, produces rapid spreading of C3-adducts across the non-self surface, coating it with C3b and its further cleavage fragments such as iC3b. On engineered nanoparticles, C3 opsonization causes 3 major problems^{[2]}:

1) C3b/iC3b avidly bind to complement receptors on phagocytes, resulting in decreased nanoparticle circulation time and increased deposition in RES organs. Indeed, for the vast majority of engineered nanoparticles, a supermajority of the injected dose ends up in the RES, instead of the target tissue^{[7]}.

2) C3b/iC3b can theoretically foul targeting moieties such as antibodies (or their fragments), inhibiting targeting of the nanoparticle to the organs and cell types of interest.

3) The C3-surface activation produces a cascade of reactions that release anaphylatoxins (C3a, C5a), producing an anaphylactoid syndrome called complement-activation-related pseudoallergy (CARPA), which includes systemic capillary leak, hypotension, and even death^{[8]}. CARPA is dose-limiting for many nanoparticle applications, and may be prohibitive for some patient populations, such as those in the intensive care unit (ICU). ICU patients are usually very sensitive to any cause of hypotension, so the sudden capillary leak and decreased blood flow caused by CARPA could be catastrophic (e.g., stroke patients would likely suffer expanded infarct volume). Thus, C3 opsonization of nanoparticles represents one of the biggest challenges for nanomedicine to realize its full potential.

Several attempts have been made to create nanomaterials that avoid C3 opsonization^{[5]}. The most frequent approach is to orthogonally append to the nanoparticle surface a “brush” coating of (usually linear) polymers. The most common of these in clinical use is polyethylene glycol (PEG), though dozens of others have shown similar or somewhat better effects. By creating a hydration shell around the particle, these polymers increase the time it takes for C3 to penetrate to the nanoparticle surface and react with surface nucleophiles. PEG and similar polymer brushes certainly do increase the time to C3 opsonization^{[9]}, increase circulation time, and decrease CARPA, but these effects are far from optimal. Even clinically-approved PEG- or dextran-coated nanoparticles suffer from eventual (within minutes) complement opsonization and CARPA^{[8]}, as elegant studies have shown that such nanoparticles rapidly develop a shell (“corona”) of physisorbed plasma proteins, and C3 then covalently bonds to these corona proteins, possibly with the corona proteins intercalated in between polymer chains^{[10,11]}. These limitations of PEG and other polymer brushes are dramatically more pronounced when targeting moieties (e.g., antibodies or their fragments) are conjugated onto the surface of nanocarriers, as the targeting moieties must extend beyond the polymer brush to engage their receptors, and this allows for C3 adduct formation. Thus, while polymer brushes are very useful and clever, they have not come close to fully winning the nano-war against complement.

### SUMMARY OF THE INVENTION

Provided herein, in a first aspect, is a composition that includes a therapeutic molecule, diagnostic molecule, molecular complex, or nanoparticle having a complement inhibitory protein attached thereto. In certain embodiments, the nanoparticle is a lipid nanoparticle or liposome. In certain embodiments, the nanoparticle is a protein-coated nanoparticle or polymeric nanoparticle. In certain embodiments, the composition includes a targeting moiety or moieties. The complement inhibitory protein may be selected from those in Table 1, or a functional variant or functional fragment thereof. In certain embodiments, the complement inhibitory protein is Factor H, Factor I, or both. In certain embodiments, the “CIP” is an aptamer or peptide that binds an endogenous CIP, such as Factor H or Factor I.

The therapeutic molecule, molecular complex, liposome, or nanoparticle may be attached to the complement inhibitory protein by a flexible linker. In certain embodiments, the liposome or nanoparticle includes a therapeutic or diagnostic molecule or multimolecular complex encapsulated therein. In certain embodiments, the therapeutic or diagnostic molecule or multimolecular complex is mRNA, miRNA, siRNA, or DNA; protein therapeutics, including antibodies, enzymes, decoy receptors; peptides; small molecule drugs; with any of these drugs functioning as anti-hypertensives, vasopressors/inotropes, pro- or anti-coagulants, anti-inflammatories, antimicrobials, immunomodulators, nutrients, anti-cancer agents (chemotherapeutics), growth factors, or inhibitors of enzymes.

In another aspect, a medical device resistant to opsonization is provided. The medical device has a complement inhibitory protein attached thereto. In certain embodiments, the medical device comprises medical tubing. In certain embodiments, the medical device comprises dialysis tubing or a membrane. In certain embodiments, the medical device comprises an implant, catheter, tubing for medical devices that contacts blood (e.g., cardiopulmonary bypass machines and hemodialysis/hemofiltration machines), functional components of medical devices that contact human blood (e.g., oxygenators used in extracorporeal membrane oxygenation; dialysis and ultrafiltration membranes used in renal replacement therapy). The complement inhibitory protein may be selected from those in Table 1, or a functional variant or functional fragment thereof. In certain embodiments, the complement inhibitory protein is Factor H, Factor I, or both. The medical device may be attached to the complement inhibitory protein by a flexible linker.

In another aspect, methods are provided. In one embodiment, the method includes attaching a complement inhibitory protein to a therapeutic molecule, molecular complex, liposome, or nanoparticle to generate a therapeutic composition capable of prolonged circulation time when administered to a subject.

In another embodiment, a method of preventing or reducing the severity of complement-activation-related pseudoallergy (CARPA) in a subject in need of therapeutic treatment is provided. The method includes administering to the subject a composition comprising a therapeutic molecule, molecular complex, liposome, or nanoparticle having a complement inhibitory protein attached thereto, wherein opsonization of the composition is prevented or reduced, thereby preventing or reducing the severity of CARPA in the subject.

In another embodiment, a method of treating in a subject in need of therapeutic treatment is provided. The method includes administering to the subject a composition comprising a therapeutic molecule, molecular complex, liposome, or nanoparticle having a complement inhibitory protein attached thereto, wherein the circulation time of the therapeutic molecule, molecular complex, liposome, or nanoparticle is increased as compared to the same therapeutic molecule, molecular complex, liposome, or nanoparticle without the complement inhibitory protein attached thereto.

Any of the compositions described herein may be used in the methods.

Other aspects and advantages of the invention will be readily apparent from the following detailed description of the invention.

### BRIEF DESCRIPTION OF THE DRAWINGS

FIG. 1A-1F. Conjugation of Factors I to nanoparticles inhibits C3-opsonization in vitro. a) Diagram of the reactions leading to C3 adducts forming on the surface of nanoparticles. The reactive thioester of C3 attacks surface nucleophiles, such as amines, resulting in a covalent C3b-surface adduct, and releasing the small protein C3a. This slow C3-surface reaction is catalytically accelerated by enzymes of the classical, lectin, and alternative pathways. C3b-surface adducts form a complex (C3bBb) that catalyzes further C3b to deposit on adjacent nucleophiles (the “amplification loop”), which allows C3b to rapidly spread across a surface. On mammalian cell surfaces, C3b is rapidly broken down by nearby regulators of complement activity (RCAs), such as Factor I, breaking the amplification loop and preventing formation of the most potent anaphylatoxin, C5a. b) Schematic of the nanoparticles on which we tested the effects of Factors H & I conjugation. All surface proteins were first conjugated to DBCO via its NHS ester, and then were conjugated onto the surface of liposomes by cycloaddition with azides on the end of the liposomes' phospholipid DSPE-PEG2k-azide. c) Clinically used nanoparticles induce complement activation in vitro. Into mouse plasma, we mixed either Abraxane (nab-paclitaxel) or liposomes conjugated to random IgG molecules (to represent nanoparticles conjugated to targeting moieties). Reactions were EDTA-quenched after 10 minutes, and C3-nanoparticle adduct formation was measured indirectly by C3a ELISA. Data is presented as the % of C3a compared to the positive control, cobra venom factor (CVF), which rapidly cleaves all C3 in solution. Compared to naive serum (no nanoparticles), both nanoparticles induced large amounts of C3a. d) Factor-I-conjugation to nanoparticles efficiently reduces C3a production. Onto IgG-liposomes, we conjugated varying amounts of Factor I (blue trace), incubated in serum using the protocol of (c), and measured C3a production. In separate experiments, we incubated IgG-liposomes with free Factor I (not conjugated to the nanoparticles; red trace). Conjugating 20 Factor I molecules onto the liposomes reduced C3a production 3.7-fold (blue box), while free Factor I molecules at a dose equivalent to 1280 Factor I per liposome led to a 27% reduction in C3a. (n=3, per condition). Inset: Comparison of C3a production by IgG liposomes with 20 Factor I per liposome (blue) vs. C3a production by IgG liposomes without Factor I or vs. C3a production by IgG liposomes with 20 albumin per liposome. Factor I induced a 73.4% reduction in C3a vs. IgG alone and a 71.0% reduction in C3a vs. irrelevant protein. e) Histogram of measurement of C3-nanoparticle surface adducts. Purified C3 was conjugated to Alexa-488, added to serum, and then nanoparticles (IgG-liposomes) were added to allow opsonization. This mixture was then flowed through a Malvern Nanosight to image and size individual nanoparticles that had become bound to fluorescent C3. The same process was performed, but for Factor I-conjugated IgG-liposomes, showing far fewer fluorescent-C3 labeled nanoparticles. FIG. 1E shows Factor I conjugation leads to far fewer fluorescent-C3-opsonized nanoparticles. Statistics: For (d), inset: n=3; *=p=2.23×10-6 for comparison of IgG+FI(20) liposomes vs. IgG liposomes, p=4.43×10-6 for comparison of IgG+FI(20) liposomes vs. IgG+Albumin(20) liposomes.

FIG. 2A-2E. Factor I conjugation decreases nanoparticle uptake by RES organs and prolongs nanoparticle circulation in the blood in mice with acute inflammation. We employed a mouse model of sepsis, IV injection of LPS, which causes neutrophils to accumulate in the lung capillaries, making the lungs become the dominant RES organ. a) Five hours after IV LPS, mice were IV-injected with 125I-labeled IgG-liposomes, +/−Factor I, and mice were sacrificed 30 minutes later to measure the biodistribution (shown as % of the injected dose per gram of tissue [% ID/g]). Factor I conjugation decreased lung uptake of the nanoparticles, while increasing blood concentration. b) The same experiment as in (a), but also showing time points 6 & 24 hours after nanoparticle injection. c & d) Pharmacokinetics of the experiment in (a-b), measuring blood concentration over time after nanoparticle injection. Factor I may slightly increase blood concentration in naive mice (c), but the increase is much bigger in sepsis-model (IV-LPS) mice (d). e) Quantification of the area-under-the-curve (AUC) of the blood concentration vs time traces, showing Factor I (striped bars) increases the AUC (for hours 0 to 6) for both naive (blue) and sepsis-model (red) mice. Statistics: For (a, b): n=6; two-way ANOVA with Sidak's correction for comparison between IgG+/−FI, and *=p<0.05, ***=p<0.001, ****=p<0.0001. For (e): n=3; comparisons between groups were made using 1-way ANOVA with Tukey's post-hoc test, all comparisons with asterisks p<0.05.

FIG. 3A-3E. Factor I conjugation prevents phagocyte association with nanoparticles, including local phagocytes in the target organ of antibody-targeted nanoparticles. a, b) In vitro, conjugation to Factor I reduces serum-treated nanoparticle interactions with neutrophils 2.0-fold. Fluorescent-IgG-liposomes+/−Factor I were incubated with serum for 1 hour, then incubated with neutrophils for 15 minutes. Neutrophils were pelleted and washed to remove unbound nanoparticles. Neutrophils were stained with the specific marker Ly6G (y-axis) and then subjected to flow cytometry. The dot-plots show Factor I changes the relative amount of liposome fluorescence seen in/on the neutrophils, which is quantified as a mean liposome-fluorescence signal (b) in the neutrophils that is half as much when Factor I is conjugated on (blue striped bar vs red striped). c) IV-LPS mice were IV-injected with 125I-liposomes conjugated to antibodies that bind to the pulmonary endothelial marker ICAM. As shown for decades, the ICAM-targeted nanoparticles homed to the lungs, but the amount of lung targeting decreased by 31.6% and the blood concentration increased (inset) by 43.7% when the liposomes were conjugated to Factor I. d) Flow cytometry of the lungs of mice similar to (c) was performed, but with liposomes traced with fluorescence instead of radioactivity. Quantification shows that Factor I led to a 21.0% lower median liposome-fluorescence in/on the lungs' local neutrophils (D-1) but a 44.6% increase in endothelial cell-associated liposome fluorescence (D-2). This decrease in neutrophil association with nanoparticles but increase in endothelial-associated nanoparticle fluorescence shows that Factor I improved the targeting specificity of ICAM-targeted liposomes. Statistics: For (b): n=6; *p<1×10-10 via two-way ANOVA with Tukey correction; ‡=p<1×10-10 via two-way ANOVA with Sidak's correction. For (c): n=3; *=p=0.0001 by two-way ANOVA with Tukey; ‡=p=0.04 by one-way ANOVA with Tukey. For (d): n=3; *=p=0.006 by two-way ANOVA with Sidak's correction; =p=0.025 by two-way ANOVA with Sidak. For (d): n=3; *=p=0.006 by two-way ANOVA with Sidak's correction; ‡=p=0.005 by two-way ANOVA with Sidak's correction.

FIG. 4A-4D. Factor I conjugation to nanoparticles prevents CARPA-associated side effects. a) Sepsis-model mice (IV-LPS) were IV-injected with IgG-liposomes+/−surface-conjugated Factor I, and sacrificed 10 minutes later for blood draw. ELISAs showed Factor I led to a nearly 50% decrease in the C3a blood concentration provoked by liposomes (left panel), and completely eliminated the production of the complement pathway's most potent anaphylatoxin C5a (right panel). b) Factor I conjugation prevented visceral organ hypoperfusion. In the same mice from (a), we observed mice receiving IgG-liposomes had very dark spleens, indicating hypoperfusion, while Factor I conjugated liposomes did not change the spleen color from its normal light burgundy. c) Treating mice the same as in (a) & (b), we measured the CBC (complete blood count) and found that IgG-liposomes markedly increase the concentration of all circulating white blood cells (WBCs; left panel), but this was largely prevented by Factor I conjugation. IgG-liposomes also increased the hematocrit (right panel), which is the most direct measure of CARPA-related systemic capillary leak. Once again, Factor I prevented this side effect of nanoparticles. d) Factor I also prevented CARPA-related cerebral hypoperfusion. Five hours after IV-LPS, mice were injected with IgG-liposomes+/−surface-conjugated Factor I, while their cerebral blood flow was measured via Doppler ultrasound of the middle cerebral artery. IgG-liposomes (red trace) led to decreased blood flow, while Factor I conjugated liposomes (blue) did not (traces are mean+/−SEM; n=3/condition). The inset shows the average rate of blood flow change per minute, which stayed fairly constant and negative for IgG-liposomes, but was consistently near zero for Factor I conjugated liposomes, showing that Factor I prevented liposome-provoked CARPA-related cerebral hypoperfusion. The average rate of blood flow change decreased by 2675% for IgG-liposomes vs IgG-liposomes conjugated to Factor I. Statistics: For (a): n=3; *p=0.0255, **p=0.0014 by Welch's t-test. For (d): n=3; *p=0.0497 by Welch's t-test.

FIG. 5. Complement activation by bare PEGylated-liposomes. Bare liposomes induced a 2.0-fold increase in C3a, relative to naive serum levels.

FIG. 6A-D. Characterization of complement protein liposomes. (a) Nanoparticle tracking analysis determination of size distributions (per size concentration represented as number of particles per mL) for liposomes with either Factor I (top panels) or Factor H (bottom panels) conjugated alongside IgG. Plots show triplicate measurement overlays for each formulation type. Leftmost panels show liposome size distributions before conjugation to proteins. Middle top panel: Liposomes with IgG and no Factor I. Right top panel: Liposomes with IgG and 20 Factor I. Middle bottom panel: Liposomes with 100 IgG and 20 Factor H. Right bottom panel: Liposomes with 200 IgG and 100 Factor H. (b-c) Sepharose column chromatography traces indicating efficiency of protein-liposome conjugation reactions. (b), left panel: Chromatography tracing 125I-Factor I binding to liposomes with IgG (IgG not traced in this data). Factor I that is not conjugated to liposomes elutes broadly from 11-18 mL. Factor I that is conjugated to liposomes elutes from 6-9 mL. Conjugation efficiency was calculated by the ratio of area under the curve of the liposome peak to the sum of the liposome peak and the free Factor I peak. Efficiency of Factor I conjugation: 30-77%, variable with respect to the number of Factor I in the conjugation reaction. (b), right panel: Chromatography tracing 125I-IgG conjugation to liposomes with Factor I (Factor I not traced in this data), as in the left panel for 125I-Factor I. IgG conjugation efficiency was >85% for both 100 and 200 IgG per liposome. (c), right panel: Chromatography tracing 125I-Factor H binding to liposomes at 2 coating densities of both IgG and Factor H. Factor H that is not conjugated to liposomes elutes broadly from 11-18 mL. Factor H that is conjugated to liposomes elutes from 6-9 mL. Conjugation efficiency was calculated by the ratio of area under the curve of the liposome peak to the sum of the liposome peak and the free Factor H peak. Efficiency of Factor H conjugation varied from 57-78%. (c), left panel: Chromatography tracing fluorescent IgG-DBCO binding to liposomes with varying amounts of Factor H and IgG. IgG not bound to liposomes elutes at ˜14 ml and IgG bound to liposomes elutes from 6-9 mL. IgG-liposome conjugation efficiency, calculated as above for Factor I and Factor H, was >95% for all quantities of Factor H and IgG. (d) Tabulated size and PDI data for unconjugated, IgG-conjugated, and IgG+Factor I- or IgG+Factor H-conjugated liposomes. Tabulated data in (d) represents average liposome size derived from analysis as in (a) over multiple preparations, with number of preparations noted in the table.

FIG. 7. Comparison of C3a generation by IgG liposomes vs. C3a generation by IgG liposomes with factor H included at 20 per liposome (left panel) or with factor I included at 20 per liposome (right panel).

FIG. 8. Factor H liposomes flocculated after conjugation.

FIG. 9. Comparison of factor I effects on C3a generation by IgG liposomes vs. irrelevant protein (albumin) effects on C3a generation by IgG liposomes. Data is identical to the inset in FIG. 1, panel D, but indicates the C3a level in naive serum by dashed line.

FIG. 10A-C. Upper panels: cartoon of human complement protein C3 represents lysines, to which Alexa Fluor 488 NHS ester can conjugate, in red. Lower panel: Spectrophotometric verification of complement protein C3 labeling with Alexa Fluor 488 NHS ester.

FIG. 11A-D. Nanoparticle tracking analysis results comparing liposome sizing and counting via light scattering in buffer (dashed lines) vs. liposome sizing and counting via surface-adsorbed C3 fluorescence (solid lines) for: (a) IgG liposomes with high IgG surface density; (b) IgG+Factor I liposomes with high IgG surface density; (c) IgG liposomes with low IgG surface density; (d) IgG+Factor I liposomes with low IgG surface density.

FIG. 12. Complement activation by IgG liposomes, with or without factor I on the liposome surfaces. The figure compares measurement of factor I effects by fluorescent C3 detection in nanoparticle tracking analysis assays with measurement of factor I effects by C3a ELISA, showing similar effects with each metric.

FIG. 13. Biodistributions of IgG liposomes in mice subject to intravenous LPS injury. The data compares IgG liposomes alone vs. IgG liposomes co-injected with factor I vs. IgG liposomes conjugated to an equivalent quantity of factor I to that provided in the co-injected dose. The data indicate that factor I extended IgG liposome circulation time and reduced non-specific lung uptake only when conjugated to the IgG liposome surface.

FIG. 14A-B. (a) Full data for biodistributions of IgG and IgG+Factor I liposomes in IV-LPS-affected mice, as represented partially in main text FIG. 2a. (b) Analogous data for comparison of IgG and IgG+Factor H liposomes in IV-LPS-affected mice.

FIG. 15A-C. (a-c) Full data for biodistributions of IgG and IgG+Factor I liposomes at different time points after liposome injection, as represented partially in main text FIG. 2b.

FIG. 16. Raw data table of FIG. 2C, blood pharmacokinetics of IgG liposomes or IgG+FI liposomes in naive or IV-LPS challenged mice. Note that in mice that received LPS (a model of sepsis), the time series plot of nanoparticle concentration in the blood shows a dip and then increase (FIG. 2d), which is quite different than the classic bi-exponential decay typical of most drugs' concentration in blood. As we showed in Myerson et al, 2021 [19], in IV-LPS mice, complement-opsonized nanoparticles also display a rapid dip in blood concentration, followed by a slower rebound (increase) in blood concentration. In that paper, we showed that the initial dip in such nanoparticles' blood concentration is due to the rapid uptake of the nanoparticles by neutrophils that are marginated in the lungs (i.e., these cells reside in the lungs' capillary lumens). After that initial dip, we showed that the rebound (increase) in blood concentration is due to a slow demargination of neutrophils (leaving the marginated state to circulate in blood), and that those demarginated neutrophils contain the nanoparticles or catabolized fragments thereof. Among our several lines of proof for this mechanism, we showed: 1) increased neutrophil numbers in the blood after nanoparticle delivery, and a decreased number of neutrophils in the lungs; 2) within the blood of these mice, we noted an increase in the relative fraction of nanoparticles (radiotraced) in the cell pellet (where circulating neutrophils reside) vs. plasma.

FIG. 17. Tabulated non-compartmental analysis parameters for IgG or IgG+FI liposome pharmacokinetics data in mice. Note that estimation of terminal slope was not valid for IgG liposomes in mice affected by intravenous LPS, due to the time traces not being monotonic, as described in FIG. 16. Non-compartmental analysis assumes monotonicity, and usually biexponential decay of plasma concentrations of nanoparticles. Therefore, parameters relying on the terminal slope estimate were not tabulated for these nanoparticles.

FIG. 18A-D. (a) Flow cytometry data depicting anti-Ly6G neutrophil stain vs. liposome fluorescence following in vitro incubation of bone marrow-derived neutrophil-enriched cell suspensions with IgG (upper panels, red) or IgG+Factor I (lower panels, blue) liposomes. Rightmost panels (dashed outlines) indicate data for liposomes treated/opsonized in serum prior to incubation with cells. (b) Data analogous to that in (a) but for liposomes with lower IgG surface density. (c) Mean liposome fluorescence in Ly6G-positive cells for the conditions outlined in (a-b), with data for bare liposomes included for comparison. (d) Data as in (c) but gated on liposome-positive neutrophils. *: p<1×10-10 for comparison of serum-treated conditions for IgG liposomes with and without Factor I. ‡: p<0.0001 for comparison of naive and serum-treated conditions for the same liposomes.

FIG. 19. Adhesion of ICAM-targeted liposomes (100 anti-ICAM mAb per liposome), with or without factor I included on the liposome surface, to ICAM-expressing REN cells. Data indicate dose-dependent adhesion of ICAM-targeted liposomes to ICAM-expressing cells, with factor I causing no change to ICAM targeting. Data for incubation of ICAM-targeted liposomes with wild-type REN cells (with no surface ICAM) is included as a negative control.

FIG. 20. Comparison of biodistribution data for ICAM-targeted vs. IgG liposomes, with or without factor I conjugated to the liposome surfaces. Data are reproduced from that depicted in FIGS. 2 and 3 in the main text, but arranged to indicate specific lung targeting for ICAM-targeted vs. IgG liposomes. *: p<1×10-10 for comparison of ICAM-targeted liposome lung uptake vs. IgG liposome lung uptake. ‡: p<1×10-10 for comparison of ICAM-targeted+FI liposome lung uptake vs. IgG+FI liposome lung uptake. Statistical significance was determined via two-way ANOVA with Tukey's correction for multiple comparisons.

FIG. 21A-B. Flow cytometry analysis of ICAM-targeted liposome distribution to neutrophils and endothelial cells in the lungs as in FIG. 3d-e in the main text. Upper panels: Anti-Ly6G neutrophil stain (left) or anti-CD31 endothelial stain vs. liposome fluorescence for single cell suspensions prepared from the lungs of mice receiving ICAM-targeted or ICAM targeted+Factor I liposomes. Lower panels: Percent of neutrophils (left) or endothelial cells (right) positive for liposome fluorescence in mouse lungs treated with ICAM-targeted or ICAM targeted+Factor I liposomes.

FIG. 22A-B. Flow cytometry analysis of ICAM-targeted liposome distribution to leukocytes in the lungs as in FIG. 3d-e in the main text. Upper panels: Anti-Ly6G CD45 leukocyte stain vs. liposome fluorescence for single cell suspensions prepared from the lungs of mice receiving ICAM-targeted or ICAM targeted+Factor I liposomes. Panels to the left indicate analysis of CD45 staining over all cells and panels to the right indicate analysis of CD45 staining over only Ly6G-negative cells (i.e., cells that are not neutrophils). Lower panels: Percent of all leukocytes (left) or leukocytes aside from neutrophils (right) positive for liposome fluorescence in mouse lungs treated with ICAM-targeted or ICAM targeted+Factor I liposomes.

FIG. 23. ELISA results measuring plasma C3a concentrations in naive mice and mice treated with intravenous LPS.

FIG. 24. Images of spleens of mice treated with IgG liposomes or IgG+factor I liposomes. All mice were treated with IV LPS five hours before liposomes.

FIG. 25. Complete blood count data from IV LPS-affected mice receiving IgG liposomes or IgG+Factor I liposomes, expanding on findings presented in FIG. 4c in the main text.

FIG. 26. Area under curve analysis of middle cerebral artery blood flow in IV LPS-affected mice receiving IgG or IgG+Factor I liposomes.

FIG. 27. Blood flow rate in the middle cerebral artery of IV LPS-affected mice after treatment with IgG or IgG+Factor I liposomes. (a) Data for liposomes with high IgG surface density. (b) Data for liposomes with lower IgG surface density.

FIG. 28-FIG. 29B demonstrate that nanoparticles conjugated to a Factor H-binding aptamer efficiently bind Factor H. FIG. 28. The aptamers were first exposed to complementary strands that quenched a fluorophore attached to the aptamer, but addition of Factor H displaces the quencher.

FIG. 29A shows effect of titration of Factor H concentrations added to double strand complex. Fluorescence increase is observed due to the release of BHQ-labeled complement strand by adding factor H to 11-nt aptamer-complement duplex. While no significant fluorescence increase is observed for adding factor H to 15-nt aptamer-complement duplex. FIG. 29B shows that the reaction is completed in a few mins.

### DETAILED DESCRIPTION OF THE INVENTION

Provided herein is a new approach to fight off C3 from the nanoparticle surface through surface conjugation of a class of proteins known as regulators of complement activation (RCA), also referred to herein as complement inhibitory proteins. Several RCAs circulate in blood and are expressed on the surface of mammalian cells, where they inhibit C3 and its upstream and downstream complement proteins. As described herein, compositions and methods are provided which incorporate complement inhibitory proteins. Perhaps the central RCA is Factor I, an 88 kDa serine protease circulating in human blood at just 35 μg/mL (compared to C3 at 1.2 mg/mL), which cleaves and inactivates C3b when Factor I is brought in close proximity to a surface^{[6,12,13]}. The nucleic acid sequence of human Factor I is reproduced in SEQ ID NO: 1, while the amino acid sequence is shown in SEQ ID NO: 2 (UniProtKB/Swiss-Prot: P05156.2).

Mammalian cells avoid complement attacking themselves by recruiting Factor I, and its soluble cofactor, Factor H, to their cell surface (along with expressing similar cofactors on their surface). The nucleic acid sequence of human Factor H is reproduced in SEQ ID NO: 3, while the amino acid sequence is shown in SEQ ID NO: 4.

To harness the power of RCAs for nanomedicine, prior studies have elegantly shown that adding high doses of RCAs to in vitro serum can reduce complement activation upon nanoparticle addition^{[14,15]}. However, clinically, co-injection of long-acting complement inhibitors with nanoparticles comes with the significant danger of systemic complement inhibition, which is a powerful immunosuppressant that is dangerous in many patient populations. A prior study non-specifically physisorbed multiple proteins, including Factor H, to silicon nanoparticles, but unfortunately this physisorption did not significantly reduce nanoparticle activation of C3 in whole serum, and failed to reduce phagocyte uptake, suggesting that Factor H is very sensitive to the method of adsorption onto nanoparticles^{[17]}.

Unless defined otherwise in this specification, technical and scientific terms used herein have the same meaning as commonly understood by one of ordinary skill in the art and by reference to published texts, which provide one skilled in the art with a general guide to many of the terms used in the present application.

As used herein, “a,” “an,” or “the” can mean one or more than one. For example, “a” cell can mean a single cell or a multiplicity of cells.

As used herein, the term “about” refers to a variant of ±10% from the reference integer and values therebetween. For example, “about” 40 base pairs, includes ±4 (i.e., 36-44, which includes the integers 36, 37, 38, 39, 40, 41, 42, 43, 44). For other values, particularly when reference is to a percentage (e.g., 90% identity, about 10% variance, or about 36% mismatches), the term “about” is inclusive of all values within the range including both the integer and fractions.

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

As used herein, the term “administered in combination” or “combined administration” means that two or more agents are administered to a subject at the same time or within an interval such that there can be an overlap of an effect of each agent on the patient. In some embodiments, they are administered within about 60 minutes, 30 minutes, 15 minutes, 10 minutes, 5 minutes, or 1 minute of one another. In some embodiments, the administrations of the agents are spaced sufficiently closely together such that a combinatorial (e.g., a synergistic) effect is achieved. In some embodiments, the administration in combination can be concurrent (i.e., all the compositions are administered as part of a single formulation, or different compositions in different formulations are administered simultaneous), or consecutive (e.g., several compositions in several formulations are administered consecutively).

The term “amino acid substitution” refers to replacing an amino acid residue present in a parent sequence (e.g., a consensus sequence) with another amino acid residue. An amino acid can be substituted in a parent sequence, for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the nomenclature ‘AnY’, wherein A is the single letter code corresponding to the amino acid naturally or originally present at position n, and Y is the substituting amino acid residue. In other aspects, substitution patterns can be described according to the nomenclature An(YZ), wherein A is the single letter code corresponding to the amino acid residue substituting the amino acid naturally or originally present at position X, and Y and Z are alternative substituting amino acid residue.

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

When used with respect to two or more moieties, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or van der Waals interactions or a hybridization-based connectivity sufficiently stable such that the “associated” entities remain physically associated.

As used herein, the term “effective amount” of an agent is that amount sufficient to effect beneficial or desired results, for example, clinical results, and, as such, an “effective amount” depends upon the context in which it is being applied. For example, in the context of administering an agent that treats a tumor, an effective amount of an agent is, for example, an amount sufficient to reduce or decrease a size of a tumor or to inhibit a tumor growth, as compared to the response obtained without administration of the agent. The term “effective amount” can be used interchangeably with “effective dose,” “therapeutically effective amount,” or “therapeutically effective dose.”

As used herein, the term “helper lipid” refers to a compound or molecule that includes a lipidic moiety (for insertion into a lipid layer, e.g., lipid bilayer) and a polar moiety (for interaction with physiologic solution at the surface of the lipid layer). Typically the helper lipid is a phospholipid. A function of the helper lipid is to “complement” the amino lipid and increase the fusogenicity of the bilayer and/or to help facilitate endosomal escape, e.g., of nucleic acid delivered to cells. Helper lipids are also believed to be a key structural component to the surface of the LNP.

The term “ionizable amino lipid” includes those lipids having one, two, three, or more fatty acid or fatty alkyl chains and a pH-titratable amino head group (e.g., an alkylamino or dialkylamino head group). An ionizable amino lipid is typically protonated (i.e., positively charged) at a pH below the pKa of the amino head group and is substantially not charged at a pH above the pKa.

As used herein, the term “identity” refers to the overall monomer conservation between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc. Unless otherwise specified, the percentage of identity values disclosed in the present application are obtained by using the implementation of MAFFT (Multiple Alignment using Fast Fourier Transform) version 7 available at the European Bioinformatics Institute (www.ebi.ac.uk/Tools/msa/mafft/with default parameters.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity “% ID” of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the term “polypeptide” refers to a polymer of amino acid residues typically joined by peptide bonds that can be produced naturally (e.g., isolated or purified) or synthetically.

“Fragments” of proteins or peptides in the context of the present invention may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide.

A fragment of a protein may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein.

Fragments of proteins or peptides may furthermore comprise a sequence of a protein or peptide as defined herein, which has a length of for example at least 5 amino acids, preferably a length of at least 6 amino acids, preferably at least 7 amino acids, more preferably at least 8 amino acids, even more preferably at least 9 amino acids; even more preferably at least 10 amino acids; even more preferably at least 11 amino acids; even more preferably at least 12 amino acids; even more preferably at least 13 amino acids; even more preferably at least 14 amino acids; even more preferably at least 15 amino acids; even more preferably at least 16 amino acids; even more preferably at least 17 amino acids; even more preferably at least 18 amino acids; even more preferably at least 19 amino acids; even more preferably at least 20 amino acids; even more preferably at least 25 amino acids; even more preferably at least 30 amino acids; even more preferably at least 35 amino acids; even more preferably at least 50 amino acids; or most preferably at least 100 amino acids.

“Variants” of proteins or peptides as defined in the context of the present invention may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g., its specific inhibitory property. “Variants” of proteins or peptides as defined in the context of the present invention may comprise conservative amino acid substitution(s) compared to their native, i.e., non-mutated physiological, sequence. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bonds, e.g., side chains which have a hydroxyl function. This means that e.g., an amino acid having a polar side chain is replaced by another amino acid having a likewise polar side chain, or, for example, an amino acid characterized by a hydrophobic side chain is substituted by another amino acid having a likewise hydrophobic side chain (e.g., serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g., using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam). A variant may also include a non-natural amino acid.

A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide.

As used herein, the term ligand (sometimes referred to herein as targeting moiety) refers to any molecule that specifically binds to another molecule, which is sometimes referred to herein as the partner molecule or target. In one embodiment, the binding moiety is an antibody. As used herein, an “antibody” is a monoclonal antibody, a synthetic antibody, a recombinant antibody, a chimeric antibody, a humanized antibody, a human antibody, a CDR-grafted antibody, a multi-specific binding construct that can bind two or more targets, a dual specific antibody, a bi-specific antibody or a multi-specific antibody, or an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one VL, one VH antigen-binding domain, and one or two constant “effector” domains optionally connected by linker domains, a univalent antibody lacking a hinge region, a single domain antibody, a dual variable domain immunoglobulin (DVD-Ig) binding protein or a nanobody. Also included in this definition are antibody mimetics such as affibodies, i.e., a class of engineered affinity proteins, generally small (˜6.5 kDa) single domain proteins that can be isolated for high affinity and specificity to any given protein target.

An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations.

The term antibody also encompasses molecules comprising an immunoglobulin domain from an antibody (e.g., a VH, CL, CL, CH1, CH2 or CH3 domain) fused to other molecules, i.e., fusion proteins. In some embodiments, such fusion protein comprises an antigen-binding moiety (e.g., an scFv). The antibody moiety of a fusion protein comprising g an antigen-binding moiety can be used to direct a therapeutic agent (e.g., a cytotoxin) to a desired cellular or tissue location determined by the specificity of the antigen-binding moiety.

### Compositions

Provided herein are compositions that include a therapeutic molecule, diagnostic molecule, molecular complex, or nanoparticle having a complement inhibitory protein (CIP), or peptide or aptamer that binds a CIP, attached thereto. These compositions are able to avoid opsonization by complement proteins, as compared to similar compositions that are not attached to CIPs.

### Complement Inhibitory Proteins

The composition includes a complement inhibitory protein (or peptide or aptamer that binds a CIP) attached to a therapeutic molecule, molecular complex, or nanoparticle. The powerful effector functions of complement have the potential to prevent therapeutic molecules from achieving their purpose in humans and other mammals. Human beings and other mammals have developed a variety of both plasmatic and membrane-bound inhibitory proteins to regulate the location and activity of complement. Herein, these proteins are referred to as complement inhibitory proteins (CIPs). Various CIPs are known in the art, including those listed in Table 1 below. See also, Zipfel and Skerka, Complement regulators and inhibitory proteins, Nature Reviews Immunology, 9(10):729-40 (October 2009), which is incorporated herein by reference. The compositions described herein utilize a complement inhibitory protein to decrease the complement response to the therapeutic component of the composition. The CIPs listed in Table 1 are useful in the compositions and methods described herein.

TABLE 1
Complement Inhibitory Proteins

| Regulator (alternate name) | Location |
|---|---|
| Factor H | Plasma |
| FHL 1 | Plasma |
| Properdin | Plasma |
| Carboxypeptidase N (Anaphylatoxin | Plasma |
| inactivator) | |
| C4BP | Plasma |
| C1q | Plasma |
| C1INH | Plasma |
| CFHR1 | Plasma |
| Clusterin (SP-40) | Plasma |
| Vitronectin (S-protein) | Plasma |
| CR1 (CD35, immune adherence receptor) | Membrane-bound |
| CR2 (CD21, Epstein-Barr virus receptor) | Membrane-bound |
| CR3 (MAC1, CD11b-CD18 and αMβ2 | Membrane-bound |
| integrin) | |
| CR4 (CD11c-CD18 and αXβ2 integrin) | Membrane-bound |
| CRIg (VSIG4) | Membrane-bound |
| CD46 (MCP) | Membrane-bound |
| CD55 (DAF) | Membrane-bound |
| CD59 (Protectin) | Membrane-bound |
| C3aR | Membrane-bound |
| C5aR | Membrane-bound |
| C5L2 | Membrane-bound |
| C1qR (CD93) | Membrane-bound |
| SIGNR1 (CD209) | Membrane-bound |

In addition to the CIPs described above, also useful herein are fragments and/or variants that retain the function of the native protein. Functional fragments and functional variants are those that retain the complement inhibitory function that is desirable for the compositions described herein. In addition, combinations of the CIPs described herein may also be utilized.

In certain embodiments, the CIP is Factor I, or a functional variant or functional fragment thereof. In other embodiments, the CIP is Factor H, or a functional variant or functional fragment thereof. In certain embodiments, the CIP includes both Factor I and Factor H, or functional variants or functional fragments thereof.

In some embodiments, rather than a CIP, a peptide or aptamer that binds to an endogenous protein that functions as the CIP, is used (referred to herein as a “peptide or aptamer that binds a CIP”). For example, in certain embodiments, the composition includes a peptide that binds a complement inhibitory protein, attached to the therapeutic molecule, molecular complex, or nanoparticle. In other embodiments, the composition includes an aptamer that binds a complement inhibitory protein, attached to the therapeutic molecule, molecular complex, or nanoparticle.

An aptamer is a short single strand DNA or RNA oligonucleotide that can fold into a three-dimensional conformation enabling the precise molecular recognition of a given target. As demonstrated herein, the DNA aptamer-conjugated nanoparticle is then able to bind an endogenous CIP, such as Factor H, that is found in the subject's blood.

In certain embodiments, the peptide or aptamer that binds a CIP binds Factor I. In other embodiments, the peptide or aptamer that binds a CIP binds Factor H. In other embodiments, the peptide or aptamer binds a CIP identified in Table 1.

In another embodiment, the aptamer binds Factor H. In certain embodiments, the aptamer has the sequence of 5′-GGT CTC GGG CAC GGG TCA GGC GGT TAT ACG GTG CCC-3′ (SEQ ID NO: 5). In another embodiment, the aptamer has the sequence of SEQ ID NO: 5 with 1, 2, 3, 4, 5, 6, 7, 8, 9, or 10 substitutions. In certain embodiments, the aptamer has the sequence of 5′-CCGACCUUAGUCAAUCACUUCGUUCGAUGAAUAGCA-3′ (SEQ ID NO: 10). In another embodiment, the aptamer has the sequence of SEQ ID NO: 10 with 1, 2, 3, 4, 5, 6, 7, 8, 9, or 10 substitutions.

In another embodiment, the aptamer binds C5aR1. In certain embodiments, the aptamer has the sequence of SEQ ID NO. 8: 5′GGAGCUCAGCCUUCACUGCGGUCUAUAUCGAGGGGGGACGAGAGGGGAUGU AUAGACCCAGGUGAGGCCUUGGGGACAAUUUGAAUCGGGCGUGGCACCACGG UCGGAUCC-3′. In another embodiment, the aptamer has the sequence of SEQ ID NO: 8 with 1, 2, 3, 4, 5, 6, 7, 8, 9, or 10 substitutions. In certain embodiments, the aptamer has the sequence of SEQ ID NO. 9-GGAGCUCAGCCUUCACUGCUAGUGACCGAUCAGGAGGGGAGAAGGAGGACAC CGUGUCAACUACGCCGUGUGAAGGAUUGCAGCUGAGGAUGGCACCACGGUCG GAUCC. In another embodiment, the aptamer has the sequence of SEQ ID NO: 9 with 1, 2, 3, 4, 5, 6, 7, 8, 9, or 10 substitutions. See, e.g., Kumar, Aptamers that bind to the human complement component receptor hC5aR1 interfere with hC5aR1 interaction to its hC5a ligand, Mol Biol Rep, Vol. 45:851-864, July 2018, which is incorporated herein by reference.

In another embodiment, the aptamer binds C4BP. In certain embodiments, the aptamer has the sequence of SEQ ID NO. 11:

UAAAGCGUCGGGCCGUACUGUCAAUACAUACAAUG. In another embodiment, the aptamer has the sequence of SEQ ID NO: 11 with 1, 2, 3, 4, 5, 6, 7, 8, 9, or 10 substitutions. In certain embodiments, the aptamer has the sequence of SEQ ID NO. 12:

GGUUUCAAUGUCCCGGUACUGUCAUCACAUACAAUG. In another embodiment, the aptamer has the sequence of SEQ ID NO: 12 with 1, 2, 3, 4, 5, 6, 7, 8, 9, or 10 substitutions. In certain embodiments, the aptamer has the sequence of SEQ ID NO. 13: GCUUAUCGUGUCUUGGUACUGUCAGCACAUACUAUG. In another embodiment, the aptamer has the sequence of SEQ ID NO: 13 with 1, 2, 3, 4, 5, 6, 7, 8, 9, or 10 substitutions. See, e.g., Fjelstrup, Soren et al. “Differential RNA aptamer affinity profiling on plasma as a potential diagnostic tool for bladder cancer.” NAR cancer vol. 4(3), zcac025. 22 Aug. 2022, Published online 2022 Aug. 22. doi: 10.1093/narcan/zcac025, which is incorporated herein by reference.

In another embodiment, the aptamer binds vitronectin. In certain embodiments, the aptamer has the sequence of SEQ ID NO. 14: AATAAACGCTCAACTCAAGTGGCGTGCGGCAGGTTGGTGTGACGGCTGGGA GGGTTCGACATG. In another embodiment, the aptamer has the sequence of SEQ ID NO: 14 with 1, 2, 3, 4, 5, 6, 7, 8, 9, or 10 substitutions. See, e.g., Stuart et al, Selection of a Novel Aptamer Against Vitronectin Using Capillary Electrophoresis and Next Generation Sequencing, Molecular Therapy—Nucleic Acids, Vol., e386, November 2016, which is incorporated herein by reference.

In another embodiment, the aptamer binds vitronectin. In certain embodiments, the aptamer has the sequence of SEQ ID NO. 15: CTCCTCTGACTGTAACCACGTTAGGCGAGAACATGTCAGTACGTCGACGTTCTAC TTGCTGCATAGGTAGTCCAGAAGCC. In another embodiment, the aptamer has the sequence of SEQ ID NO: 15 with 1, 2, 3, 4, 5, 6, 7, 8, 9, or 10 substitutions. See, e.g., Zamay et al, Aptamers Selected to Postoperative Lung Adenocarcinoma Detect Circulating Tumor Cells in Human Blood, Molecular therapy: the journal of the American Society of Gene Therapy vol. 23(9):1486-96, September 2015., which is incorporated herein by reference.

The number of CIPs or peptide or aptamer that binds a CIP, per therapeutic molecule, diagnostic molecule, molecular complex, or nanoparticle may be varied. In certain embodiments, at least 5, at least 10, at least 20, at least 25, or at least 30 CIPs (or peptides/aptamers) are present per therapeutic molecule, molecular complex, or nanoparticle. In certain embodiments, about 10 to about 30 CIPs (or peptides/aptamers) are present per therapeutic molecule, molecular complex, or nanoparticle. In certain embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 CIPs (or peptides/aptamers) are present per therapeutic molecule, molecular complex, or nanoparticle. In certain embodiments, where more than one CIP is utilized, the number of CIPs (or peptides/aptamers) per therapeutic molecule, diagnostic molecule, molecular complex, or nanoparticle may be individually selected.

The compositions described herein include a therapeutic molecule, diagnostic molecule, molecular complex, and/or nanoparticle.

### Nanoparticles

In certain embodiments, the composition includes a nanoparticle. Nanoparticles are materials with overall dimensions in the nanoscale, i.e., under 100 nm. In recent years, these materials have emerged as important players in modern medicine, with applications ranging from contrast agents in medical imaging to carriers for gene delivery into individual cells. Nanoparticles have a number of properties that distinguish them from bulk materials simply by virtue of their size, such as chemical reactivity, energy absorption, and biological mobility. Various types of nanoparticles are known in the art, and the term includes all nanoparticles useful in the medical arts. Many potential applications of nanomedicine have been, or are being, explored, including nanoparticle-based molecular imaging probes for biological studies and disease detection; nanocarriers for targeted in vivo drug/gene delivery for more efficient therapy; nanoparticles as direct therapeutic agents; and nuclease-based biological nanomachines for genome editing.

Two classes of nanoparticles together represent a plurality of clinically-approved nanomedicine therapeutics: lipid-based nanoparticles and protein-coated nanoparticles. In certain embodiments, the nanoparticle is a lipid nanoparticle. In certain embodiments, the lipid nanoparticle is a liposome. Other useful nanoparticles include quantum dots, gold nanoparticles, magnetic nanoparticles, and superparamagnetic iron oxide nanoparticles. See, Murthy, S K, Nanoparticles in modern medicine: State of the art and future challenges, Int J Nanomedicine. 2007 June; 2(2): 129-141, and Tong et al, Nanomedicine: tiny particles and machines give huge gains. Ann Biomed Eng. 2014 February; 42(2):243-59. Epub 2013 Dec. 3, which are incorporated herein by reference.

Lipid nanoparticles have been described for use in various types of therapy including those for delivery of vaccines (e.g., SARS-CoV-2/COVID-19), cancer, neurodegenerative disease, HIV/AIDS, ocular diseases, respiratory diseases, e.g., for targeted delivery by surface functionalization, for prolonging residence time in vivo, for solubilizing water-insoluble drugs, for transport across blood-brain barrier (e.g., by PEG incorporation), for gene therapy in diseases unresponsive to small molecule drugs, etc. The term “lipid nanoparticle”, also referred to as LNP, refers to a particle having at least one dimension on the order of nanometers (e.g., 1-1,000 nm) which includes one or more lipids (e.g., cationic lipids, non-cationic lipids, and PEG-modified lipids). In some embodiments, such lipid nanoparticles comprise a cationic lipid and one or more excipient selected from neutral lipids, charged lipids, steroids and polymer conjugated lipids (e.g., a pegylated lipid).

In one embodiment, the LNP is a liposome. Liposomes are defined as phospholipid vesicles consisting of one or more concentric lipid bilayers enclosing discrete aqueous spaces. The unique ability of liposomal systems to entrap both lipophilic and hydrophilic compounds enables a diverse range of drugs to be encapsulated by these vesicles. Hydrophobic molecules are inserted into the bilayer membrane, and hydrophilic molecules can be entrapped in the aqueous center. Furthermore, the large aqueous center and biocompatible lipid exterior permits the delivery of a variety of macromolecules, such as DNA, proteins and imaging agents. As a drug delivery system, liposomes offer several advantages including biocompatibility, capacity for self-assembly, ability to carry large drug payloads, and a wide range of physicochemical and biophysical properties that can be modified to control their biological characteristics. Liposomal formulations are characterized by properties such as particle size, charge, number of lamellae, lipid composition, and surface modification with polymers and ligands—these all govern their stability in vitro and in vivo. Encapsulation within liposomes protects compounds from early inactivation, degradation and dilution in the circulation. They consist of a lipid bilayer that can be composed of cationic, anionic, or neutral lipids and cholesterol, which encloses an aqueous volume. As used herein, the term lipid nanoparticle includes liposomes.

In the context of the present invention, lipid nanoparticles are not restricted to any particular morphology, and should be interpreted as to include any morphology generated when a cationic lipid and optionally one or more further lipids are combined, e.g., in an aqueous environment and/or in the presence of a nucleic acid compound. For example, a liposome, a lipid complex, a lipoplex and the like are within the scope of a lipid nanoparticle. An LNP may comprise any lipid capable of forming a particle to which the CIP, peptide, or aptamer that binds a CIP binds, and/or in which the one or more therapeutic molecules are encapsulated.

In certain embodiments, the lipid nanoparticle may comprise any cationic or ionizable lipid, i.e., any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2dimyristyloxyprop-3-yl)-N,N-dimethyl-N-hydroxyethyl ammonium bromide (DMRIE). Other useful lipids include, without limitation, 98N12-5, C12-200, PLGA, PEG, PEG-DMG, PEGylated lipids, amino alcohol lipids, and KL22.

Additionally, a number of commercial preparations of cationic lipids are available which can be used in the present invention. These include, for example, LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 4-(dimethylamino)-butanoic acid, (10Z,13Z)-1-(9Z,12Z)-9,12-octadecadien-1-yl-10,13-nonadecadien-1-yl ester (DLin-MC3-DMA), N,N-dimethyl-2,2-di-(9Z,12Z)-9,12-octadecadien-1-yl-1,3-dioxolane-4-ethanamine (DLin-KC2-DMA). See also, e.g., WO2014/089486, US 2018/0353616A1, and U.S. Pat. No. 8,853,377B2, which are incorporated by reference.

In certain embodiments, lipid nanoparticle formulation is performed using routine procedures comprising cholesterol, ionizable lipid, helper lipid, PEG-lipid and polymer forming a lipid bilayer around an encapsulated therapeutic molecule, e.g., encapsulated mRNA (Kowalski et al., 2019, Mol. Ther. 27(4):710-728). In some embodiments, LNP comprises a cationic lipid (i.e. N-[1-(2,3-dioleoyloxy)propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino)esters (PBAEs). In some embodiments, the LNP comprises C14-4/DOPE/Chol/PEG-lipid. See, Rybakova Y., Kowalski P. S., Huang Y., Gonzalez J. T., Heartlein M. W., DeRosa F., et al. (2019). mRNA delivery for therapeutic anti-HER2 antibody expression in vivo. Mol. Ther. 27, 1415-1423 which is incorporated by reference. In other embodiments, the LNP comprises L319/DSPC/Chol/PEG-DMG. See, Thran M., Mukherjee J., Pönisch M., Fiedler K., Thess A., Mui B. L. (2017). mRNA mediates passive vaccination against infectious agents, toxins, and tumors. EMBO Mol. Med. 9, 1434-1447 which is incorporated by reference. See also, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference.

In certain embodiments, the lipid nanoparticle comprises one or more additional lipids which stabilize the formation of particles during their formation. Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-lcarboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3phosphocholine (DSPC).

In some embodiments, the lipid nanoparticles comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In various embodiments, the lipid nanoparticles further comprise a steroid or steroid analogue. In certain embodiments, the steroid or steroid analogue is cholesterol. In some of these embodiments, the molar ratio of the cationic lipid to cholesterol ranges from about 5:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the lipid nanoparticle comprises glycolipids (e.g., monosialoganglioside GM1).

In some embodiments, the lipid nanoparticles comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the lipid nanoparticle comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3di(tetradecanoxy)propyl)carba mate or 2,3-di(tetradecanoxy)propyl-N—(ω-methoxy(polyethoxy)ethyl)carbamate. In various embodiments, the molar ratio of the cationic lipid to the pegylated lipid ranges from about 100:1 to about 25:1.

In certain embodiments, an aptamer that binds a CIP is formulated with the LNP itself. For example, the aptamer may be modified to have 3′ cholesterol, with or without a spacer. The aptamers are mixed with a lipid mixture in organic solvent, such as chloroform or ethanol. Liposomes or other LNPs are then produced using routine procedures. See, e.g., Sung, T C., Chen, W Y., Shah, P. et al. A replaceable liposomal aptamer for the ultrasensitive and rapid detection of biotin. Sci Rep 6, 21369 (February 2016), which is incorporated herein by reference.

In other embodiments, the LNP is conjugated to the CIP, or peptide or aptamer that binds a CIP. For example, the aptamer may be functionalized with DSPE-PEG, and subsequently conjugated to prepared LNPs. See, e.g., Liang, C., Guo, B., Wu, H. et al. Aptamer-functionalized lipid nanoparticles targeting osteoblasts as a novel RNA interference-based bone anabolic strategy. Nat Med 21, 288-294 (February 2015), which is incorporated herein by reference. In another embodiment, the CIP is modified with DBCO and conjugated to azide-functionalized liposomes. Methods of producing lipid nanoparticles are well known in the art. Other exemplary lipid nanoparticles and their manufacture are described in the art, for example in U.S. Patent Application Publication No. U520120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

In one embodiment, the nanoparticle is a protein nanoparticle, also referred to as a protein-coated nanoparticle. In general, protein nanoparticles offer many advantages, such as biocompatibility and biodegradability. Moreover, the preparation of protein nanoparticles and the corresponding encapsulation process involves mild conditions without the use of toxic chemicals or organic solvents. Protein nanoparticles can be generated using proteins, such as fibroins, albumin, gelatin, gliadine, legumin, 30Kc19, lipoprotein, and ferritin proteins, and are prepared through emulsion, electrospray, and desolvation methods. See, e.g., Hong S, et al. Protein-Based Nanoparticles as Drug Delivery Systems. Pharmaceutics. 2020 Jun. 29; 12(7):604. doi: 10.3390/pharmaceutics12070604. PMID: 32610448; PMCID: PMC7407889, which is incorporated herein by reference.

In one embodiment, the nanoparticle is a polymeric nanoparticle. Polymer-based nanoparticles are colloidal systems made up of natural or synthetic polymers. They furnish certain advantages over other nanocarriers such as liposomes, micelles and inorganic nanosystems, and include the feasibility of scale-up and the manufacturing process under Good Manufacturing Practices (GMP). Other peculiar characteristics of polymeric nanoparticles are the significant stability of polymeric nanoparticles in biological fluids along with the wide availability of various polymers, the opportunity to functionalize their surfaces and to modulate polymer degradation and the leakage of the entrapped compound(s) as a function of specific stimuli. Ideally, the polymers selected for parenteral administration must be biocompatible, biodegradable, and possess specific mechanical and physicochemical properties. Biodegradable polymers include synthetic polymers such as poly(D,l-lactide) (PLA), poly(D,L-glycolide) (PLG), co-polymer poly(lactide-co-glycolide) (PLGA), polyalkylcyanoacrylates, poly-ε-caprolactone. See Gagliardi et al, Biodegradable Polymeric Nanoparticles for Drug Delivery to Solid Tumors, Front. Pharmacol., 3 Feb. 2021 https://doi.org/10.3389/fphar.2021.601626, which is incorporated herein by reference.

### Therapeutic Molecules, Diagnostic Molecules, and Multimolecular Complexes

In certain embodiments, in addition to, or instead of, a nanoparticle, the composition includes a therapeutic or diagnostic molecule, or multimolecular complex. The therapeutic or diagnostic molecule, in some embodiments, is directly attached to the CIP. In other embodiments, the therapeutic or diagnostic molecule is encapsulated in an LNP. In other embodiments, the therapeutic or diagnostic molecule is conjugated to the LNP. In certain embodiments, LNP is bound to a targeting moiety. In other embodiments, the therapeutic or diagnostic molecule comprises the targeting moiety. That is, in certain embodiments, the CIP is attached directly to the targeting moiety.

Diagnostic and therapeutic agents include, without limitation, contrast agents for imaging, including CT-, MRI-, and nuclear-contrast agents; mRNA, miRNA, siRNA, or DNA; protein therapeutics, including antibodies, enzymes, decoy receptors; peptides; small molecule drugs; with any of these drugs functioning as anti-hypertensives, vasopressors/inotropes, pro- or anti-coagulants, anti-inflammatories, antimicrobials, immunomodulators, nutrients, anti-cancer agents (chemotherapeutics), growth factors, inhibitors of enzymes, and other IV-injected therapeutics. In one embodiment, the therapeutic agent is mRNA. In another embodiment, the agent is a contrast agent such as a chelated MRI or nuclear agent.

Other non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

In certain embodiments, the therapeutic or diagnostic agent includes a radionuclide. In certain embodiments, the molecular complex or LNP includes a radionuclide.

In certain embodiments, the therapeutic molecule, diagnostic molecule, molecular complex, or nanoparticle is associated with a targeting moiety that binds to a target on the surface of the target cell, e.g., a tumor cell, epithelial cell, immune cell, etc. In certain embodiments, the ligand is an antibody or an antigen binding fragment thereof, and the target is an antigen on the surface of the target cell. In other embodiments, the target is a cell surface receptor, and the targeting moiety is its cognate ligand. Representative targets include endothelial cell markers such as ICAM-1, PECAM, CD13, CD29, CD34, CD36, CD44, CD47, CD61, E-selectin, GMP-140, CD86, CD80, CD93, ICAM-2, endoglin, VCAM-1, nectin, c-Kit, CD121a, thrombomodulin, tissue factor, ACE, ACE2, VE-cadherin, MCAM, CD147, CD151, CD160, EPCR, CD213a, CD248, VEGFR2, ADAMs 8, 9, 10, 12, 15, 17, and 33, ADAMTS-13, ADAMTS-18, CXCL16, DCBLD2, endomucin, ESAM, FABP, IgG, Integrin a4/i1, KLF4, LYVE-1, notch, podocalyxin, podoplanin, RLIP76, stabilin-1, stabilin-2, TEM8, THSD1, Tie-1, Tie2, TNAP, TNF RII, VE-cadherin, VE-statin, VG5Q, and VWF. Goncharov N V et al. Markers and Biomarkers of Endothelium: When Something Is Rotten in the State. Oxid Med Cell Longev. 2017; 2017:9759735. Epub 2017 Nov. 23, which is incorporated herein by reference. Other representative targets include tumor specific antigens and tumor associated antigens such as CD20, HER2, CD19, PSA, TRP-2, EpCAM, GPC3, mesothelin (MSLN), EGFR, CEA, MUC1, AFP, tyrosinase, MAGE, and CD28.

### Medical Devices

Certain medical devices are known to trigger complement activation, when they come into contact with mammalian blood in vivo. For example, complement has been known to be activated during dialysis for over four decades, however this area of research has been neglected in recent years. Despite significant progress in biocompatibility of hemodialysis membranes and peritoneal dialysis fluids, complement activation remains an undesired effect and relevant issue. Thus, in another aspect, a medical device resistant to opsonization is provided. The medical device has a CIP or peptide or aptamer that binds a CIP, attached thereto.

In certain embodiments, the medical device is hemodialysis (HD) membrane. Patients receiving dialysis are often afflicted with an incompatibility reaction that is the result of complement activation by the membrane and closely resembles the pseudo-anaphylactic clinical picture known as complement activation-related pseudoallergy (CARPA). Complement activation takes place in the plasma (the fluid phase), but also on surfaces (the solid phase). In addition to fluid phase activation, complement depositions have also been shown on the surface of the HD membranes.

In another embodiment, the medical device comprises tubing that contacts blood. Such applications include tubing for cardiopulmonary bypass machines and hemodialysis/hemofiltration machines.

In another embodiments, the medical device comprises a functional component of a larger device that contact human blood. Such applications include oxygenators used in extracorporeal membrane oxygenation and dialysis and ultrafiltration membranes used in renal replacement therapy.

Other medical devices include stents, shunts, surgical drains, catheters (such as central venous catheters) and the like.

Methods of applying coatings to medical devices are known in the art for uses such as non-fouling coatings and eluting drugs. See, e.g., Glumac et al, An Anti-Fouling Airway Stent, Proceedings of the 2022 Design of Medical Devices Conference, Apr. 11-14, 2022, which is incorporated herein by reference.

In certain embodiments, the medical device encapsulates or releases a therapeutic or diagnostic molecule or multimolecular complex.

### Linker

In certain embodiments, the medical device, therapeutic molecule, diagnostic molecule, molecular complex, or nanoparticle is attached to the CIP or peptide or aptamer that binds a CIP, by a linker. In certain embodiments, the linker is a flexible linker. In certain embodiments, the linker is a PEG linker. PEGs are highly flexible linear or branched polymers in the 0.4-40 kDa MW range, synthetized with different end-groups. One of the end-groups is used for covalent attachment to free carboxy, amino, or sulphydryl groups on macromolecules, on LNPs or on linkers that bind the PEG to LNPs, such as phosphatidylethanolamine, via one of a variety of chemically reactive functional groups (acrylate, methacrylate, maleimide, dibenzocyclooctynol, vinyl sulfonate or vinyl or allyl ethers). The other end-group is most frequently a methyl group (methoxy-PEG), although hydroxy (—OH), amino (—NH3+), butoxy (—O—(CH2)3)-CH3) and tert-butoxy (—O—(CH3)3) terminal endings are also used. In one embodiment, the linker is PEG2000. In other embodiments, the linker is PEG400, PEG3350, PEG6000, or PEG5000. Other suitable linkers are known in the art. In certain embodiments, the linker has a molecular weight from about 500 Da to about 5000 Da. In certain embodiments, the linker is from about 5 to about 100 molecular units in length.

### Pharmaceutical Compositions

Provided herein, in one aspect, is a pharmaceutical composition comprising a therapeutic molecule, diagnostic molecule, molecular complex, or nanoparticle having a complement inhibitory protein, or a peptide or aptamer that binds a CIP, attached thereto, and a carrier, excipient, or diluent. In certain embodiments, the nanoparticle is a lipid nanoparticle or liposome. In other embodiments, the nanoparticle is a protein-coated nanoparticle or polymeric nanoparticle. In certain embodiments, the composition further includes a targeting moiety or moieties. The CIP may be selected from those described herein, e.g., from those of Table 1. In certain embodiments, the CIP is Factor H. In other embodiments, the CIP is Factor I.

The composition may be suspended in a physiologically compatible carrier to be administered to a subject in need thereof. In certain embodiments, for administration to a human patient, the composition is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. For intravenous delivery, a pH of about 6.8 to about 7.2 may be desired. However, other pHs within the broadest ranges and these subranges may be selected for other route of delivery.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer.

In another embodiment, the composition includes a carrier, diluent, excipient and/or adjuvant. Suitable carriers may be readily selected by one of skill in the art. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water.

Optionally, the compositions may contain other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

In one embodiment, a composition includes a final formulation suitable for delivery to a subject, e.g., is an aqueous liquid suspension buffered to a physiologically compatible pH and salt concentration. In another embodiment, the composition may be transported as a concentrate which is diluted for administration to a subject. In other embodiments, the composition may be lyophilized and reconstituted at the time of administration.

The compositions are administered in sufficient amounts to achieve provide a therapeutic benefit without undue adverse effects, or with medically acceptable physiological effects, which can be determined by those skilled in the medical arts. Routes of administration include direct delivery to a desired organ (e.g., the liver (optionally via the hepatic artery), lung, heart, eye, kidney), intratumoral, oral, inhalation, intranasal, intratracheal, intraarterial, intraocular, intravenous, intramuscular, subcutaneous, intradermal, and other parental routes of administration. Routes of administration may be combined, if desired.

Dosages of the composition will depend primarily on factors such as the condition being treated, the age, weight and health of the patient, and may thus vary among patients. For example, a therapeutically effective human dosage of an LNP is generally in the range of from about 10 to about 1000 microliters to about 100 mL of solution containing concentrations of from about 0.1 to about 30 mg/kg subject weight including all integers or fractional amounts within the range, and preferably 1 mg to about 700 mg liposomes for a human patient (of about 70 kg average body weight). In certain embodiments, the dosage is about 1 to about 10 mg/kg. In certain embodiments, the dosage is about 1 to about 5 mg/kg. In certain embodiments, the dosage is about 0.5 to about 5 mg/kg. In certain embodiments, the dosage is about 0.1 to about 10 mg/kg. All ranges include endpoints and all numbers between the range.

In certain embodiments, the composition is administered to a subject in a single dose. In certain embodiments, composition may be delivered via multiple injections (for example 2 doses).

### Methods

The compositions described herein are useful for preventing or reducing the severity of complement-activation-related pseudoallergy (CARPA) in a subject in need of therapeutic treatment. In certain embodiments, the method includes administering to the subject a composition comprising a therapeutic molecule, molecular complex, liposome, or nanoparticle having a complement inhibitory protein, or a peptide or aptamer that binds a CIP, attached thereto. Opsonization of the composition is prevented or reduced (as compared to a similar composition that does not comprise complement inhibitor protein), thereby preventing or reducing the severity of CARPA in the subject. The composition is such that it treats the condition for which the subject requires therapeutic treatment.

In addition to preventing or reducing CARPA, in certain embodiments, the circulation time of the therapeutic molecule, molecular complex, liposome, or nanoparticle is increased as compared to the same therapeutic molecule, molecular complex, liposome, or nanoparticle without the complement inhibitory protein (or peptide or aptamer that binds a CIP) attached thereto. Thus, in another aspect, a method of generating a composition capable of prolonged circulation time in a subject is provided. The method includes attaching a complement inhibitory protein to a therapeutic molecule, molecular complex, liposome, or nanoparticle to generate a therapeutic composition capable of prolonged circulation time when administered to a subject as compared to the same therapeutic molecule, molecular complex, liposome, or nanoparticle without the complement inhibitory protein attached thereto. Any of the compositions described herein may be utilized in the methods.

### EXAMPLES

The following examples are illustrative only and are not a limitation on the invention described herein.

### Example 1: Conjugation to Factors H & I Dramatically Decreases C3-Nanoparticle Adduct Formation

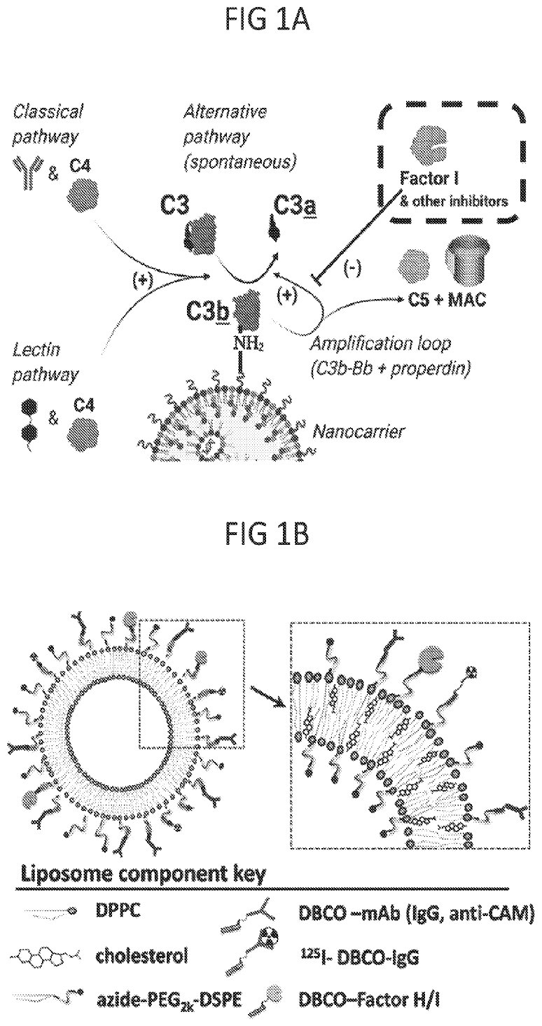

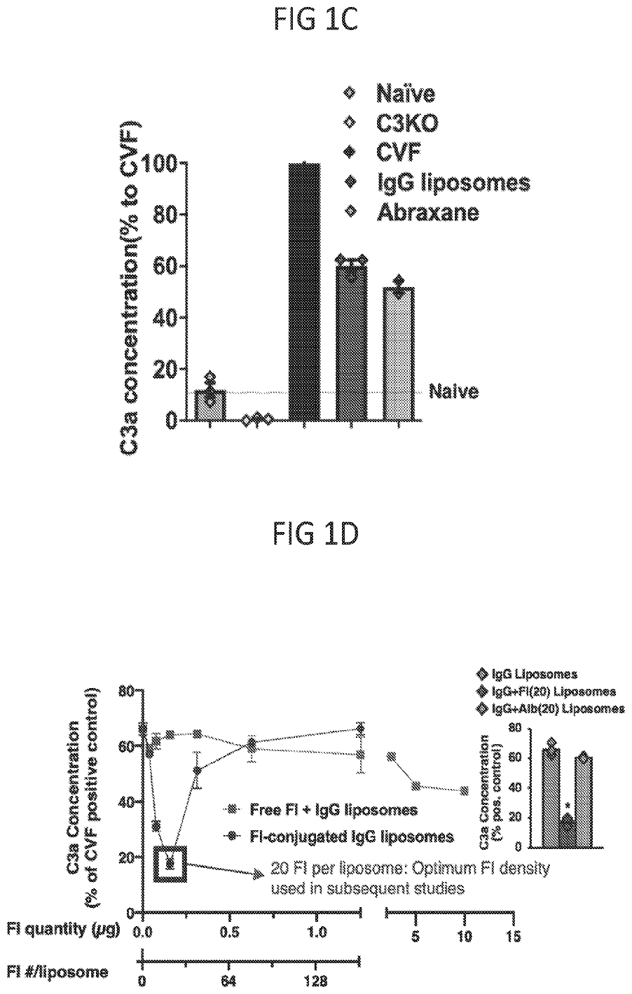

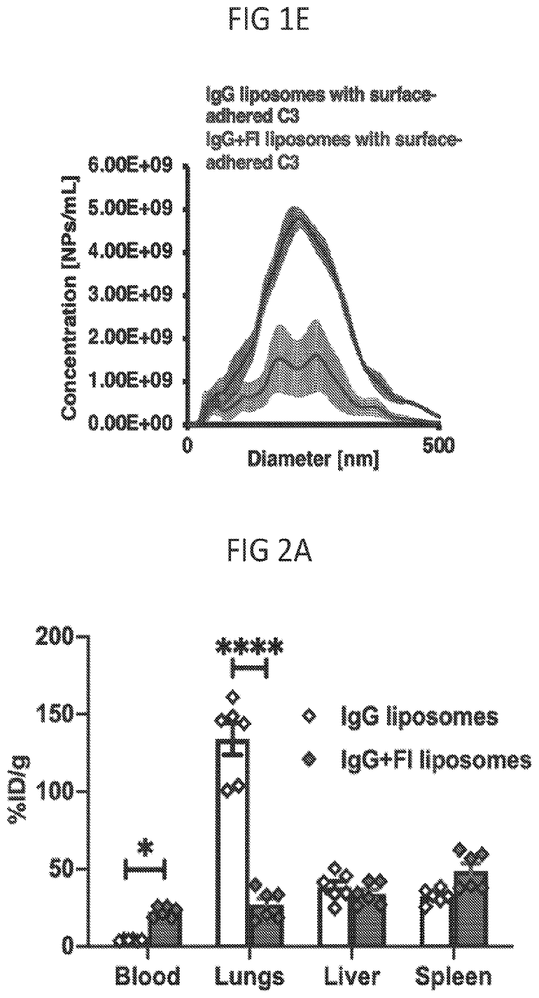

Within the enormous space of nanomaterials, we chose to focus on the two classes of nanoparticles which together represent a plurality of clinically-approved nanomedicine therapeutics: lipid-based nanoparticles (liposomes and lipid nanoparticles/LNPs) and protein-coated nanoparticles. We began by measuring the extent to which C3-surface adducts form on these nanomaterials' surfaces. Into in vitro mouse serum, we added either the one clinically approved protein nanoparticle, Abraxane (130 nanometer albumin particles loaded with paclitaxel) or a liposome conjugated to a targeting moiety (here, just random IgG). We quenched the reaction with EDTA (complement enzymes are Ca²⁺-dependent) after 10 minutes. To quantify C3-surface adduct formation, we employed the classic technique of measuring, by ELISA, the release of the small protein fragment C3a into the bulk solution. Note that C3a production is precisely stoichiometric with C3-surface adduct formation: when one C3 molecule covalently bonds to a surface nucleophile, it releases one C3a, and leaves one C3b covalently bound to the surface, usually via an amide or ester linkage. As shown in FIG. 1c, both Abraxane and IgG-liposomes increase C3-surface adduct formation, by 5.1-fold (p<0.0001) and 4.4-fold (p<0.0001), respectively. Thus, these nanoparticles elicit C3a levels that are not far below that of the positive control, cobra venom factor (CVF), which cleaves all the available soluble C3 to release C3a, and thus serves as a ceiling for the maximal amount of C3 that can react. Notably, PEG-coated liposomes without surface-conjugated IgG induced only a 2.0-fold increase in C3a relative to naive serum, underscoring the role of surface-conjugated targeting moieties in provoking complement activation (FIG. 5). Thus, for testing strategies to inhibit complement opsonization of nanoparticles, Abraxane can serve as a facile test material that is FDA-approved, and IgG-liposomes can represent the numerous targeted nanocarriers that have been brought to clinical studies.

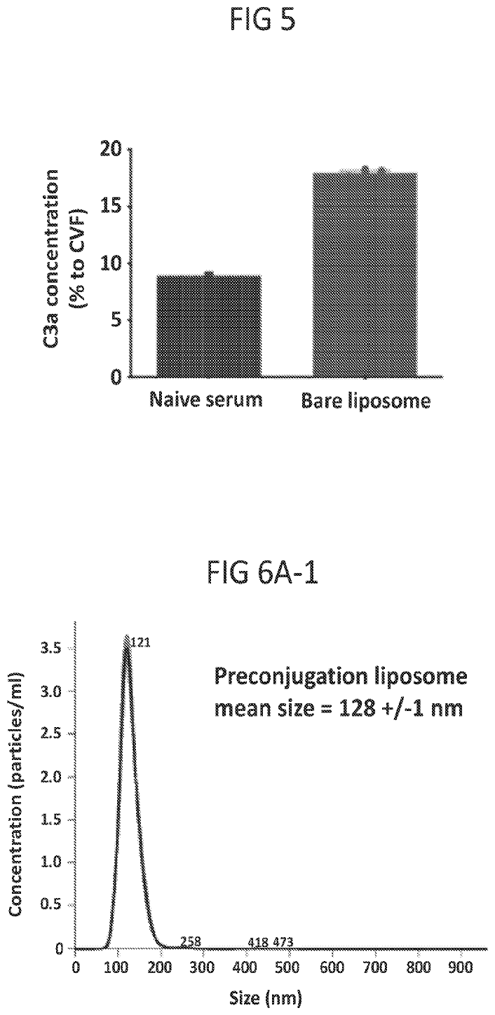

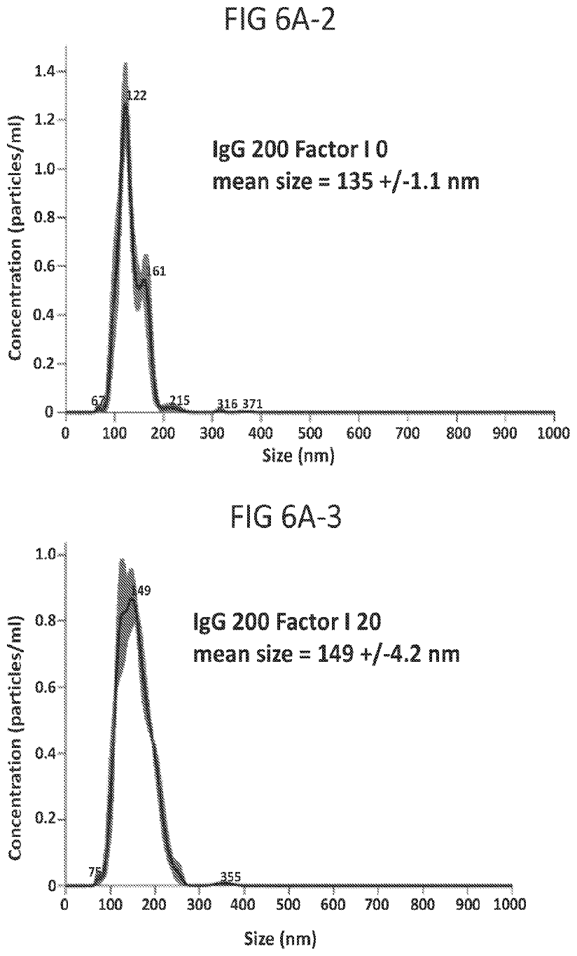

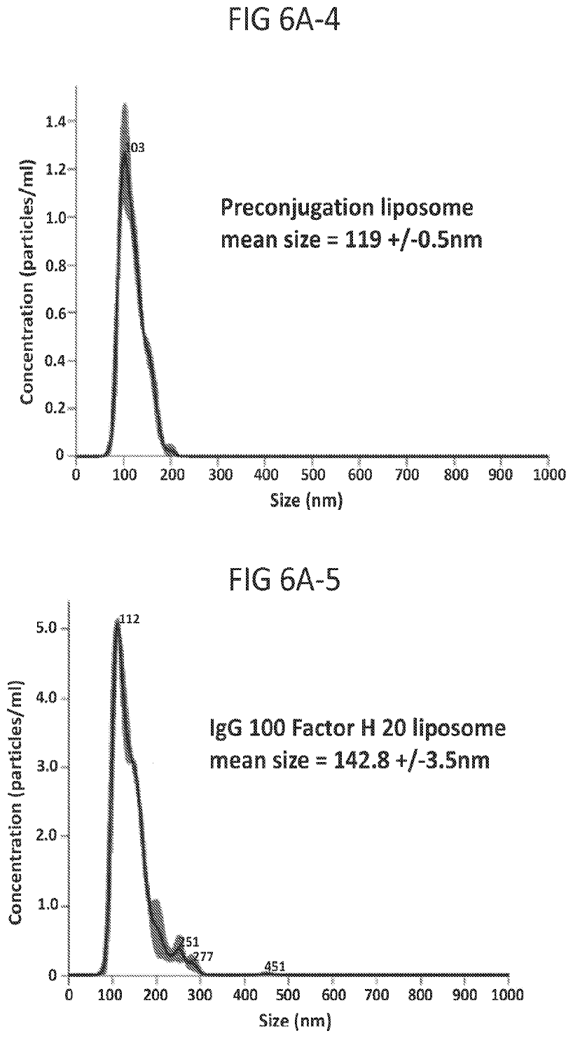

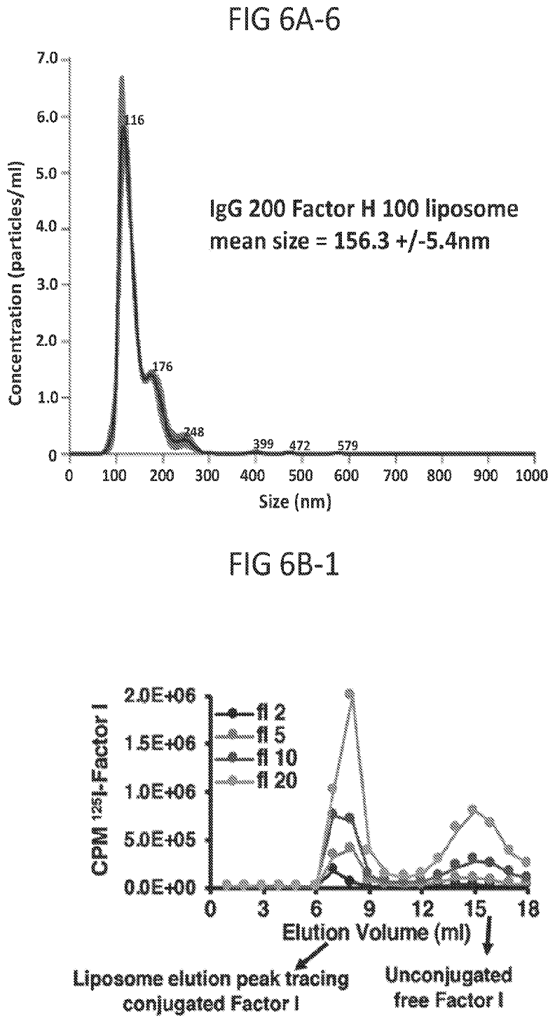

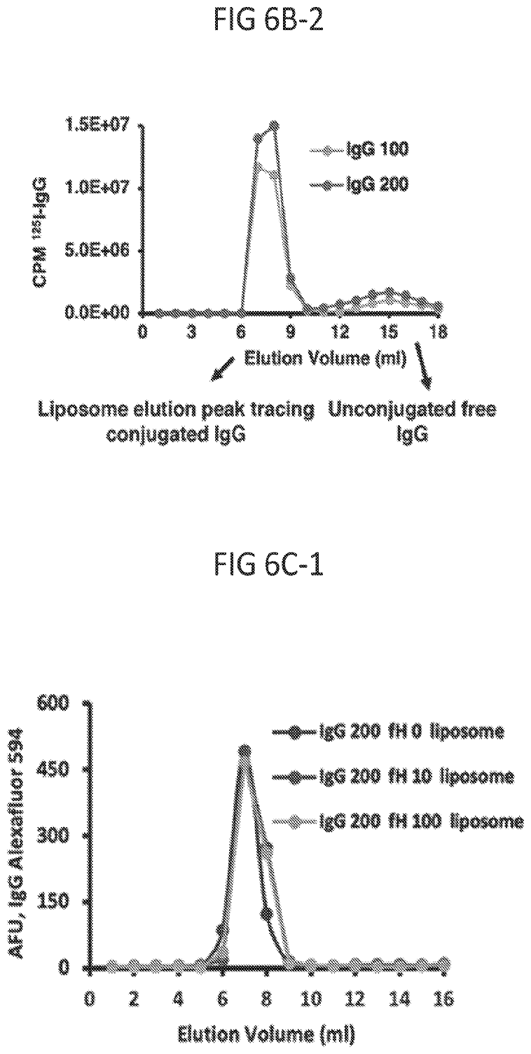

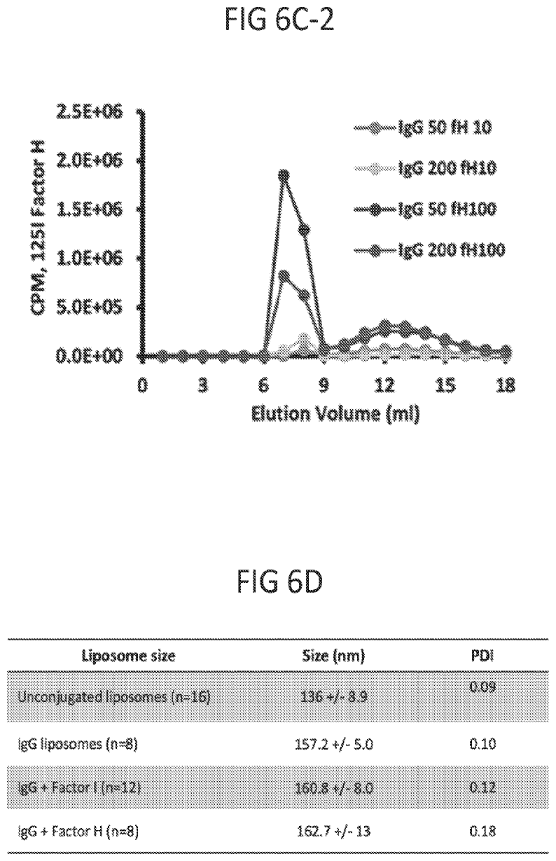

Next, to test our core hypothesis about the utility of conjugating RCAs to nanoparticles, we constructed liposomes that possessed on their surface not just a targeting moiety, but also Factors H or I (FIG. 1b). We used a standard liposome formulation composed of the lipids DPPC and cholesterol (similar to clinical products like Doxil and Arykace), and also low molar fractions of lipids linked to PEG-2000 (linear PEG of molecular weight 2 kDa), end-capped by azide for bioorthogonal conjugation chemistry. Importantly, these long PEG chains are currently the field's standard for preventing nanoparticle opsonization, allowing us to determine if Factors H & I can improve beyond the current state of the art. To the surface azides, we employed strain-promoted-alkyne-azide cycloaddition (SPAAC; a copper-free “click chemistry”) to conjugate on select proteins which had previously been conjugated to the alkyne-containing linker dibenzocyclooctyne (DBCO)^{[18]}. In this manner, we conjugated IgG molecules (random or anti-ICAM IgG; sometimes ¹²⁵I-labeled for radiotracing) and varying numbers of Factor I molecules. FIG. 6A-D provides materials characterization of all the key nanoparticles including naive unconjugated liposomes (136+/−8.9 nm), IgG/mAb conjugated liposomes (144+/−9.5 nm) and those containing complement proteins Factor H (154+/−13 nm) or I (143+/−8.9 nm) in addition to IgG including size (z-average, by dynamic light scattering), polydispersity index, and the efficiency of conjugation to IgG and Factor I. Additionally, FIG. 6A-D shows example traces from the gel exclusion chromatography used to purify the protein-conjugated liposomes away from unconjugated protein and quantify extent of conjugation of bound moieties.

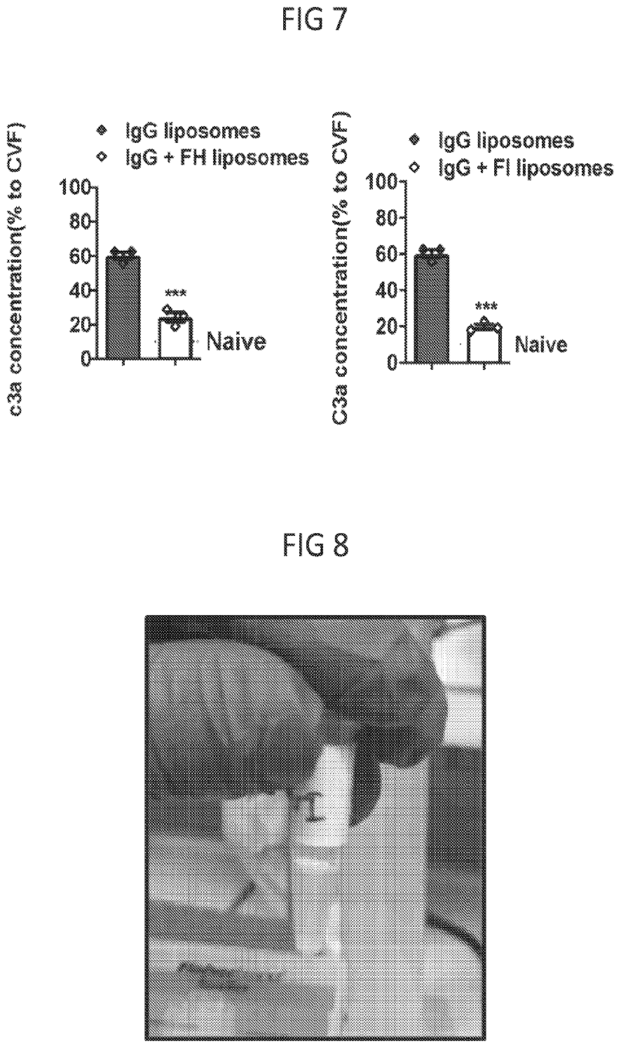

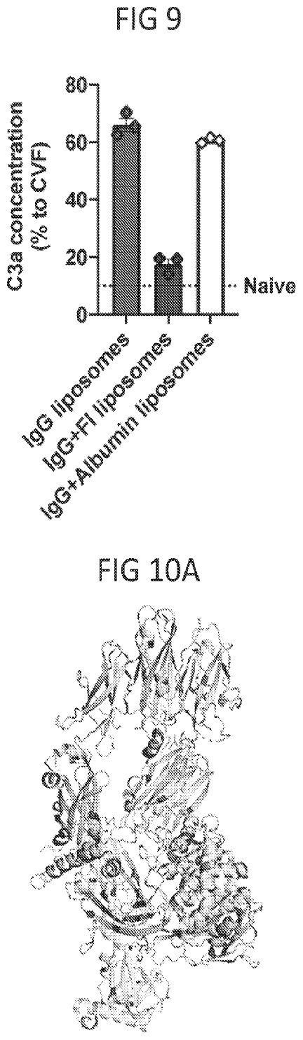

After mixing these nanoparticles with serum in vitro, we then measured C3-adduct formation using the above C3a ELISA. FIG. 1d (blue trace) shows that the conjugation of Factor I onto the nanoparticle surface efficiently decreases the production of C3-nanoparticle adducts, with the optimal Factor I surface concentration (20 Factor I molecules/liposome) reducing C3a levels by 3.7-fold (p<0.0001). By contrast, adding free Factor I (not conjugated to the nanoparticle surface) to the serum had minimal effect, with 1,280 free Factor I molecules required to reduce the C3a production by just 27% (FIG. 1d, red trace). We created similar particles with Factor H instead of Factor I, and achieved similar suppression of C3a production (FIG. 7). However, while Factor-I-conjugated nanoparticles were very stable in terms of size and PDI for up to 1 week, Factor H nanoparticles have a tendency to flocculate, with ˜50% of batches flocculating (aggregating to a macroscopic size and settling out) at 24 hours at 4° C. (FIG. 8). Therefore, for subsequent experiments, we used 20 molecules of Factor I per liposome. As further controls, we replaced Factor I with albumin, and found no reduction C3a levels (FIG. 1D, inset, FIG. 9). Thus, these experiments show that Factor I conjugation onto nanoparticles efficiently prevents complement opsonization.

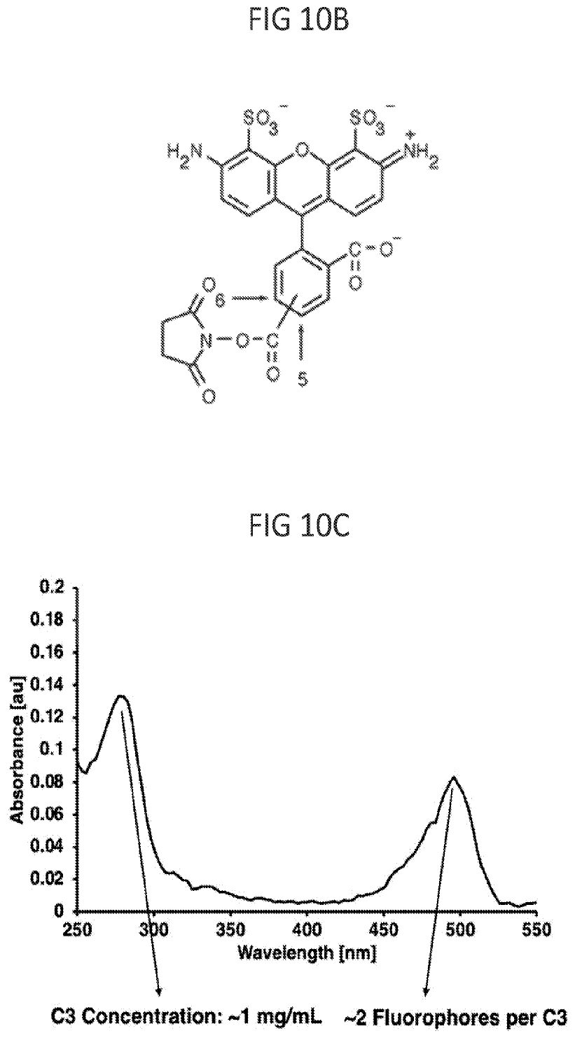

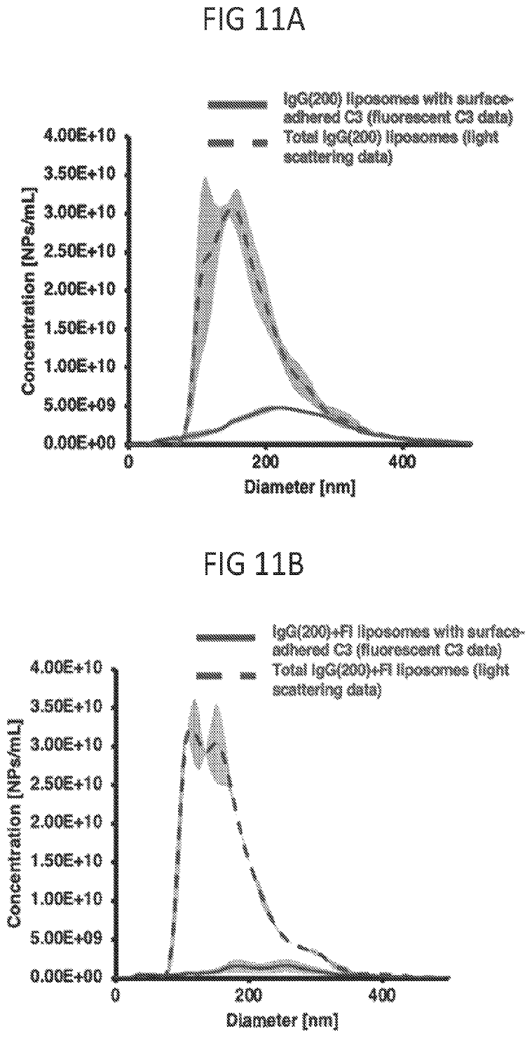

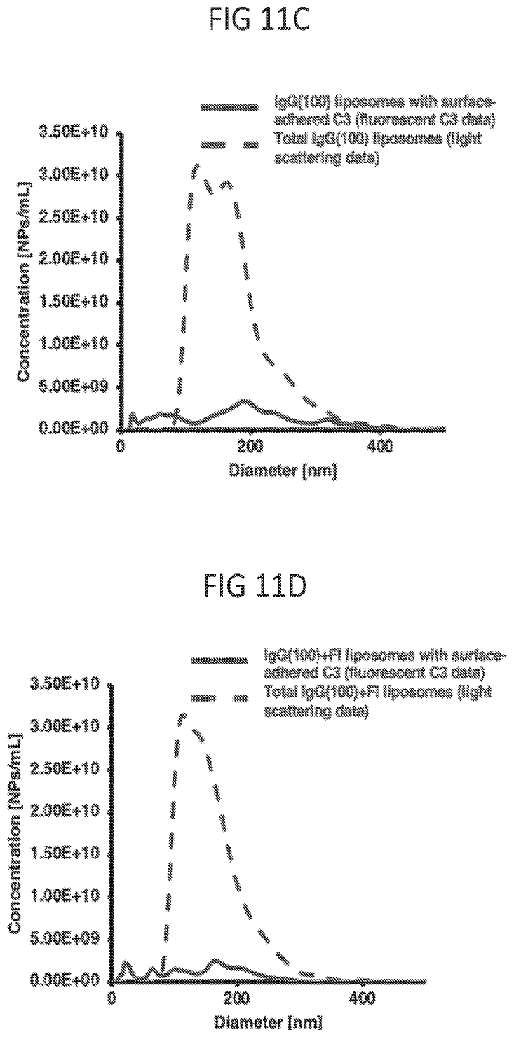

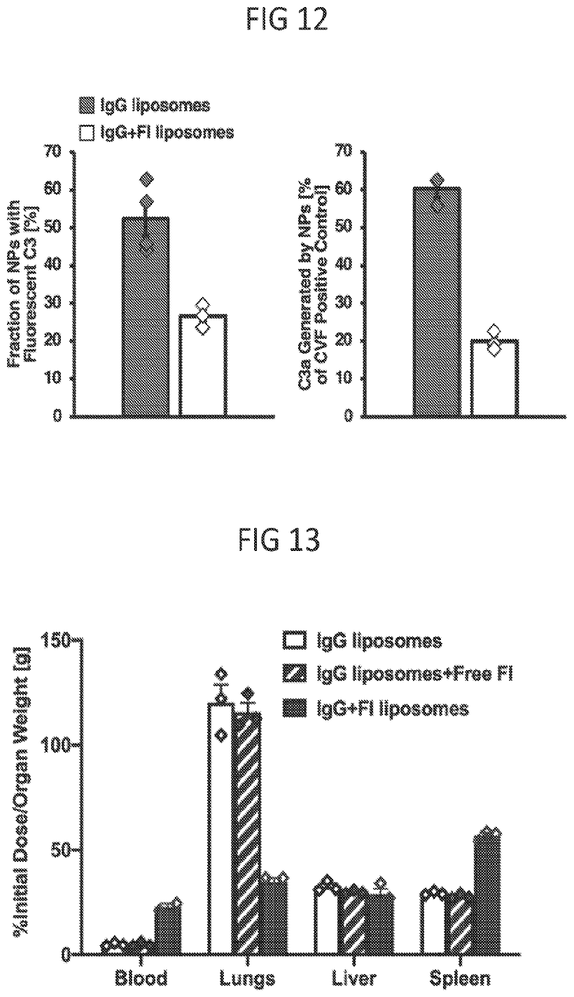

To further confirm this result, we developed a new assay of C3-nanoparticle adduct formation. The classical C3a ELISA technique is, of course, an indirect measurement of C3-nanoparticle adduct formation, as it is detecting release of a soluble product, not the adducts themselves. Therefore, we sought to directly quantify C3-nanoparticle adduct formation. To do this, we conjugated a small-molecule fluorophore to the surface of purified C3 (FIG. 10A-C), which we added to mouse serum, and then mixed in nanoparticles for 20 minutes, followed by EDTA-quenching. We then injected diluted nanoparticles-in-serum suspensions, under continuous flow, into a nanoparticle tracking analysis (NTA) device (Malvern NanoSight) to detect C3-fluorescence on individual IgG-liposomes and count and size the nanoparticles with detectable C3-fluorescence (FIG. 1E). As expected, nanoparticles with detectable fluorescent C3 are slightly larger than the nanoparticles before incubation with serum (FIG. 11A-D). Having established this first-ever direct visualization of C3-adducts on nanoparticles, we measured the C₃-fluorescence signals on IgG-liposomes that also had Factor I conjugated to their surface. Factor I clearly reduced the number of nanoparticles that had detectable C3-fluorescence (FIG. 1E), to a degree closely resembling the Factor I effect in C3a measurements of complement activation (FIG. 12). Thus, this novel NTA-based measurement shows directly that conjugation of RCA proteins like Factor I to the nanoparticle surface prevents C3-nucleophile surface adduct formation.

In summary, these results confirm that, in vitro, C3-surface adducts form rapidly and extensively on clinically used nanoparticles, but conjugation of RCA proteins (here Factors H & I) markedly reduces the C3-surface reaction. We showed this both via the classical indirect assay of a C3a ELISA, and a newly introduced NTA-based direct measurement of C3-surface adducts.

### Factor I Surface-Conjugation Prevents RES-Organ Uptake of Nanoparticles in Mouse Models of Disease, Dramatically Improving the Pharmacokinetics and Biodistribution

We next sought to investigate how Factor H & I conjugation onto nanoparticles could affect in vivo pharmacokinetics and biodistribution. To investigate this in an animal model of disease, we sought a mouse model in which complement has already been shown to drive changes in biodistribution. Previously it has been shown that in mouse models of sepsis (dysregulated immune response to infection), global complement activation is increased^{[19,20]}, and most notably for the present study, the capillaries of the lungs fill with neutrophils, which then avidly take up circulating microbes and protein-containing nanoparticles via a C3-dependent mechanism^{[21-23]}. Thus, during sepsis, the lungs are a dominant RES organ in humans and rodents. In primates, rodents, and a few other mammals, the lungs are not dominant RES organs in healthy individuals (the liver dominates), but in acute inflammatory states, such as sepsis and pneumonia, neutrophils and other leukocytes appear in enormous numbers in the lung capillaries, making the lung temporarily the dominant RES organ, as it is at all times in pigs, sheep, and most other mammals^{[24-28]}. Therefore, we chose to study Factor I's ability to change lung uptake (a proxy for complement-dependent RES uptake) in a mouse model of sepsis, in which mice are IV-injected with lipopolysaccharides (LPS) 5 hours before nanoparticle injections.

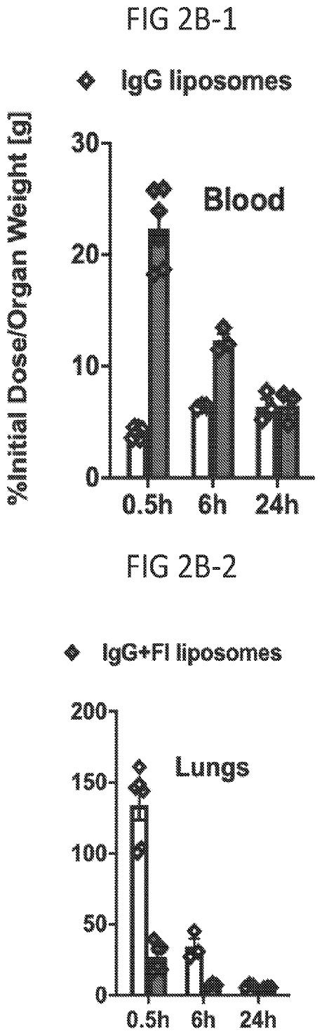

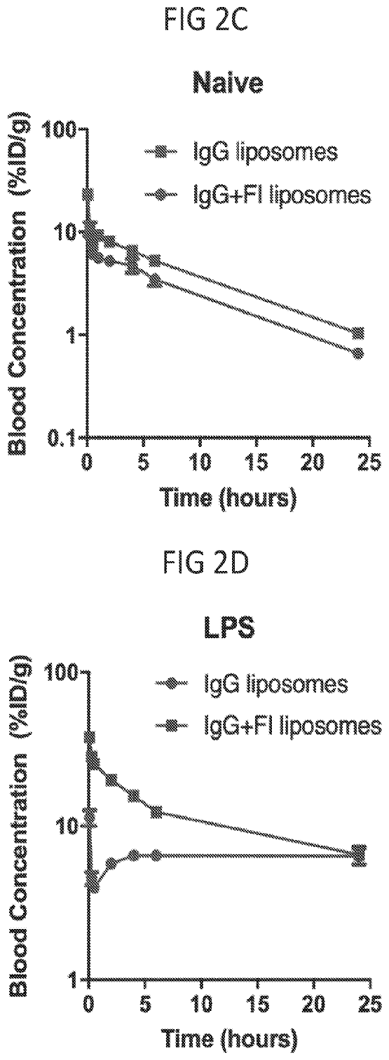

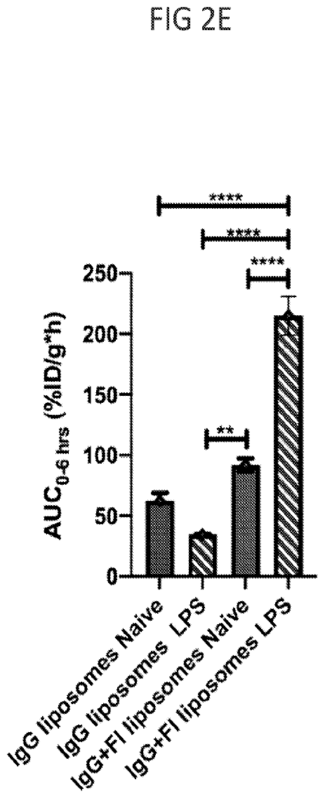

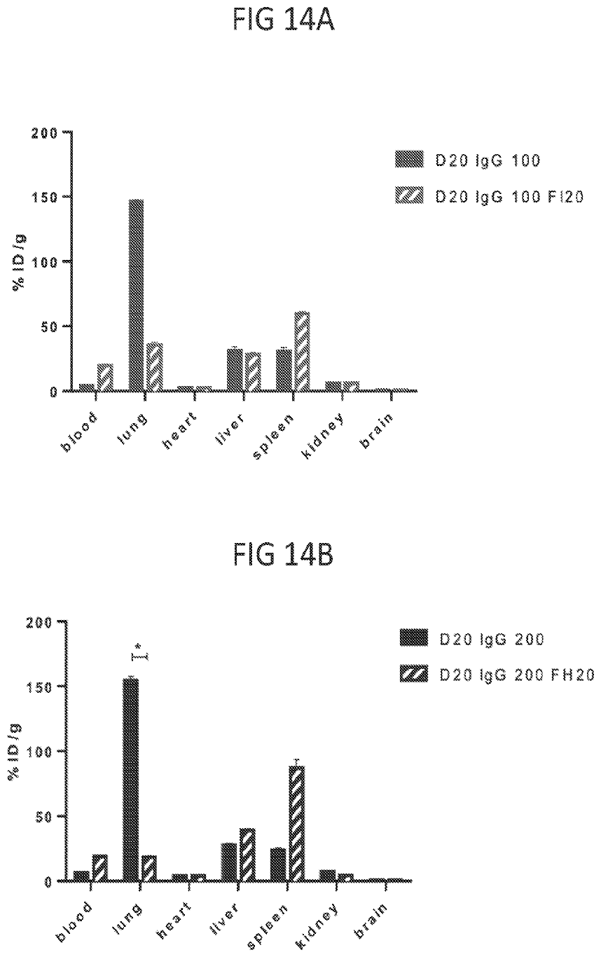

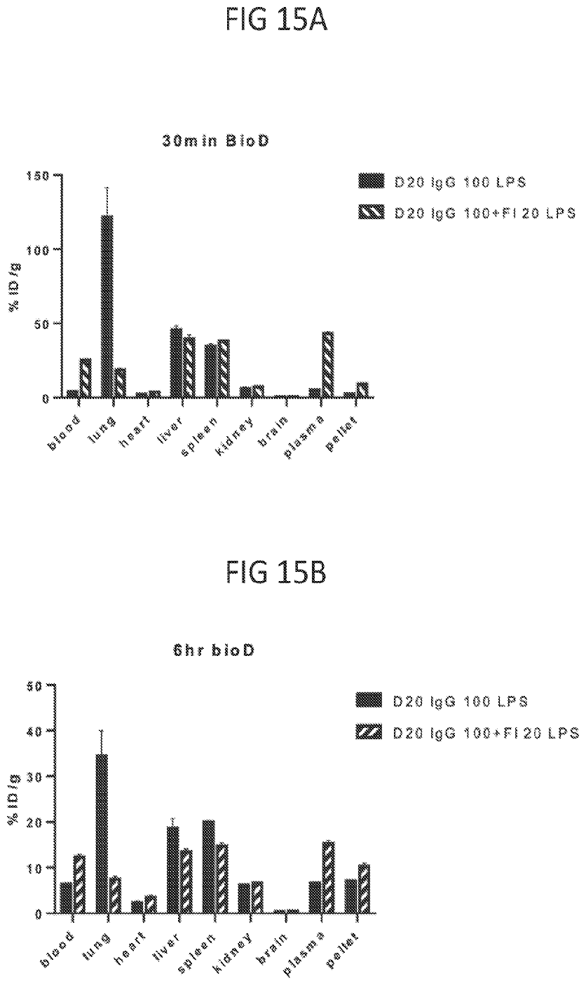

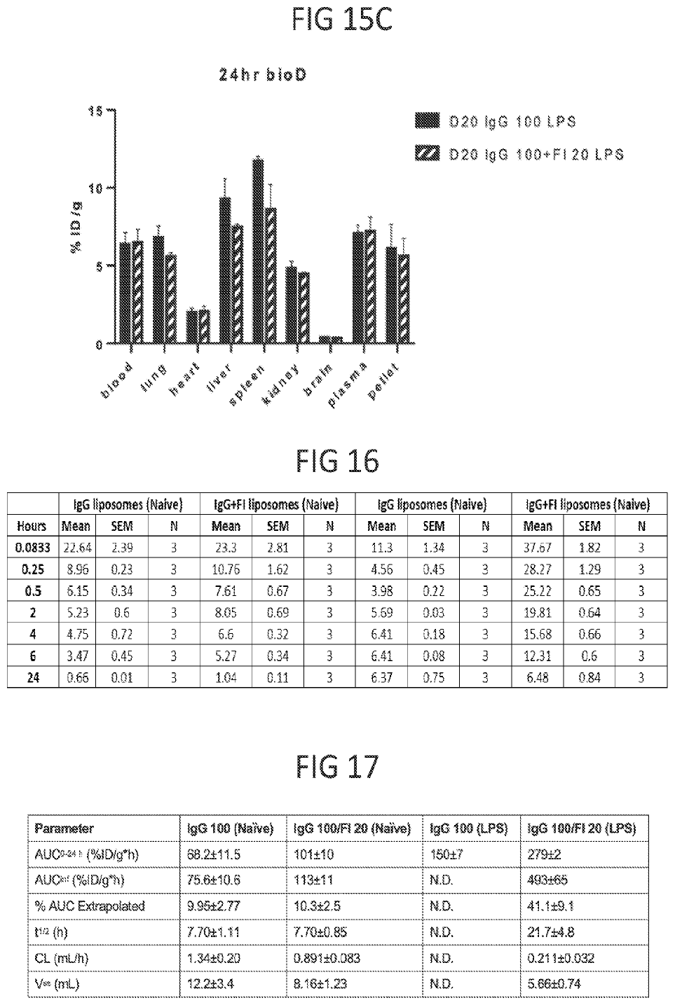

Into these sepsis-model mice, we IV-injected ¹²⁵I-labeled IgG-liposomes, plus or minus surface-conjugated Factor I. As shown in the biodistribution (obtained via gamma counter) 30 minutes after nanoparticle injection (FIG. 2a), the nanoparticles with Factor I are taken up in the lungs at lower concentrations (reduction of 5.0-fold, p<0.0001). Decreased organ uptake in the dominant RES organ (lungs) should lead to higher blood concentrations, which indeed is the case (FIG. 2a). Notably, these biodistribution changes induced by nanoparticle-conjugated Factor I were not observed when co-injecting nanoparticles with free Factor I (not conjugated to nanoparticles; FIG. 13). This trend of higher blood concentrations and lower lung/RES concentrations of Factor I nanoparticles persists at 6 hours, but is mostly gone by 24 hours (FIG. 2b; FIG. 14A-B & FIG. 15A-C), which may be because the lung's neutrophils migrate out of the pulmonary capillaries after the sepsis stimulus (LPS) clears. Thus, at least in this major model of disease, Factor I conjugation can powerfully prevent RES uptake of nanoparticles.

Having observed these changes in biodistribution, we next wanted to precisely quantify the effects of Factor I conjugation on nanoparticle pharmacokinetics. As shown in FIG. 2c, even in naive mice, Factor I-conjugated nanoparticles display higher blood concentrations over time (see also FIG. 16). In sepsis-model mice (LPS), the improvement in the blood-concentration vs time curve is much greater (FIG. 2d). In the sepsis-model mice, the area-under-the-curve (AUC; for hours 0-6) of the blood-concentration vs time curve is 6.2-fold higher (p<0.0001) in Factor I-conjugated nanoparticles than in the same particles without Factor I (FIG. 2e; see FIG. 16-17 for non-compartmental analysis and an explanation of why the concentration of nanoparticles is not monotonically decreasing over time). Thus, Factor I conjugation is able to markedly improve the pharmacokinetics of nanoparticles, in situations in which the goal is to maintain the nanoparticle circulating for a long time.

### Factor I Surface-Conjugation Prevents Nanoparticle Uptake by Local Phagocytes, Thus Improving Targeting to the Cell Type of Interest

We next sought to determine if Factor I conjugation could aid with targeting a cell type of interest. We hypothesized that Factor I conjugation would prevent uptake of the targeted nanoparticles by local phagocytes in the target organ, thus encouraging uptake in other cell types. We tested this hypothesis using a nanoparticle-targeting strategy that has been used to target the endothelial cells of the lungs for decades: anti-ICAM antibodies conjugated to nanoparticles^{[29-31]}.

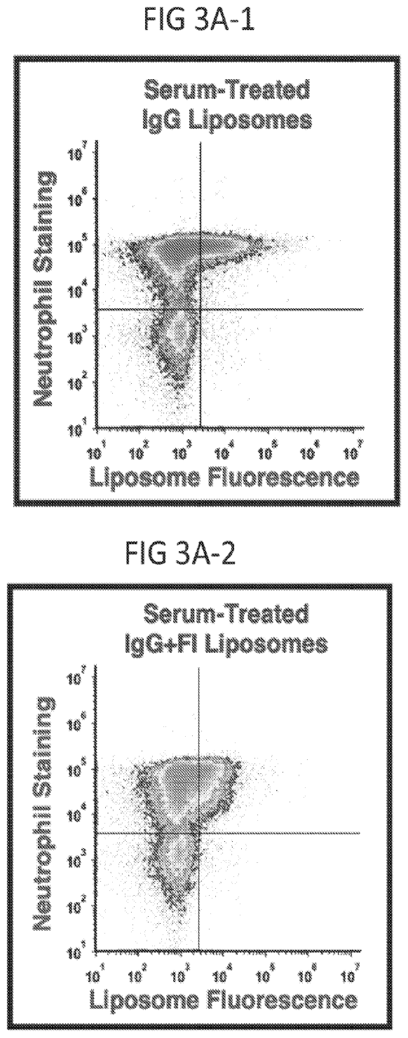

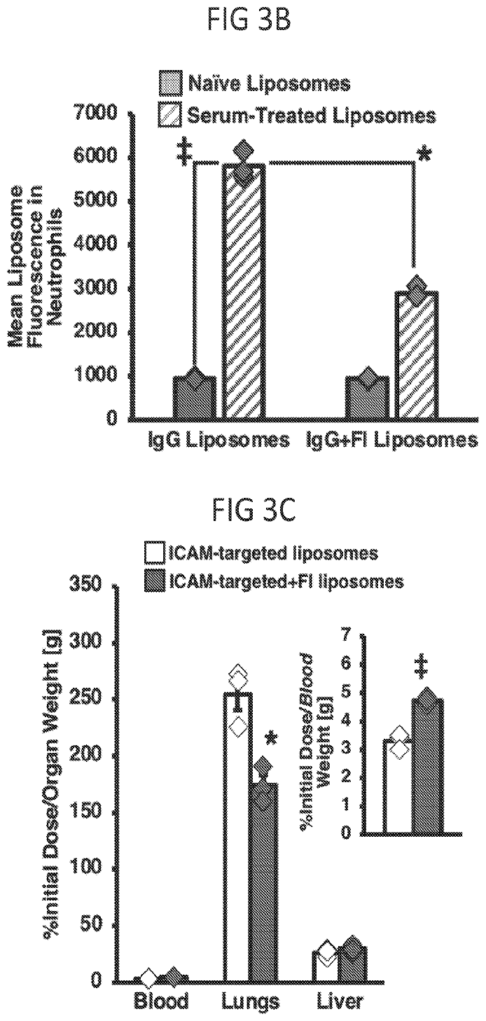

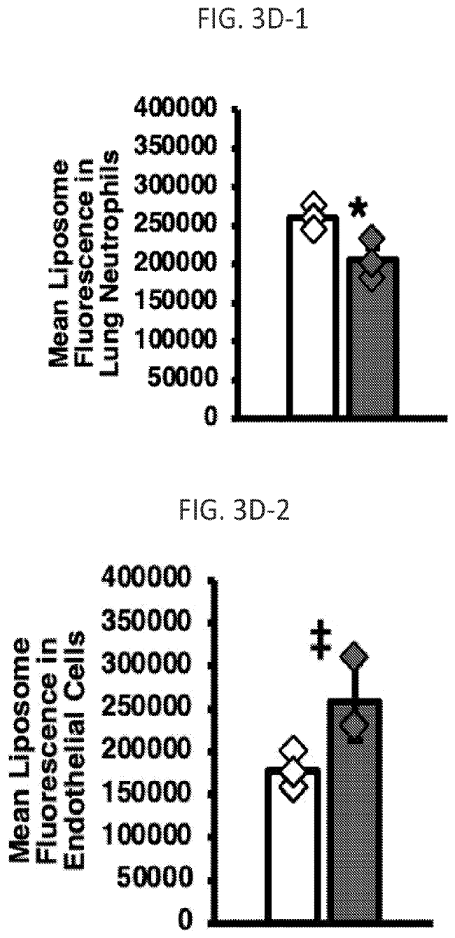

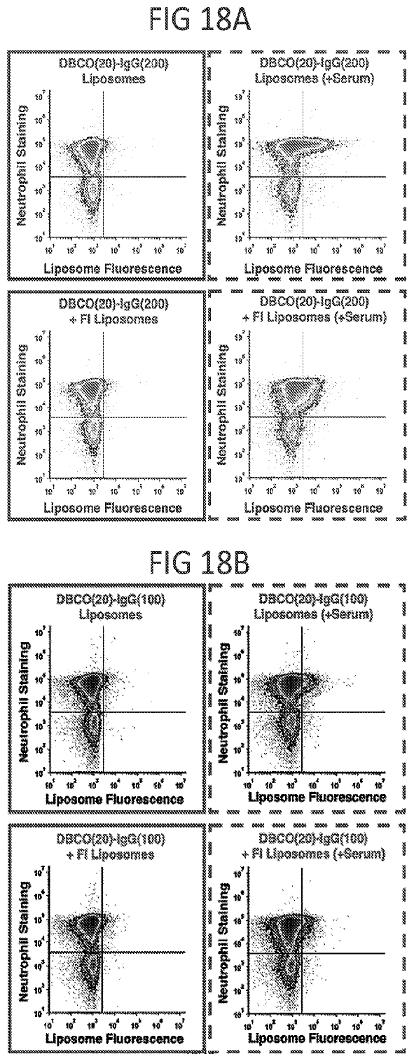

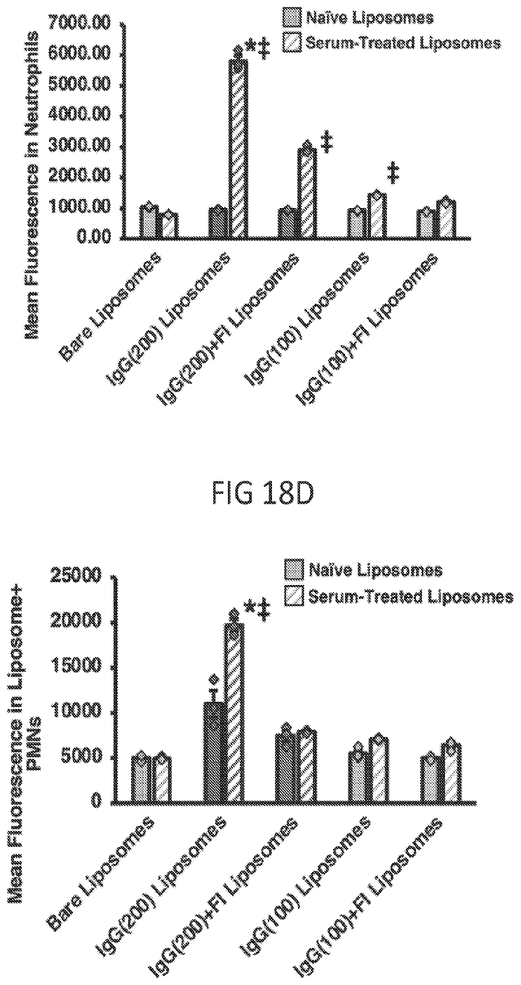

We began by first confirming that, in vitro, Factor I conjugation could prevent uptake of nanoparticles by phagocytosis. We incubated fluorescently-tagged IgG-liposomes, plus or minus Factor I conjugation, with mouse serum for 1 hour to allow for maximal C3 deposition. We then incubated the serum-exposed nanoparticles with freshly isolated mouse neutrophils rotating at 37° C. for 15 minutes. We next ran the neutrophils in a flow cytometer, assaying for the amount of liposome fluorescence associated with each neutrophil. As shown in FIG. 3a, with Factor I, there were far fewer neutrophils that displayed very high amounts of nanoparticle fluorescence. Additionally, the median nanoparticle fluorescence in/on neutrophils was decreased by 2.0-fold (p<1×10⁻¹⁰) in the Factor I conjugated nanoparticles (FIG. 3b; also FIG. 18A-D). Thus, even under stringent in vitro conditions, Factor I managed to inhibit complement-mediated association with phagocytes.

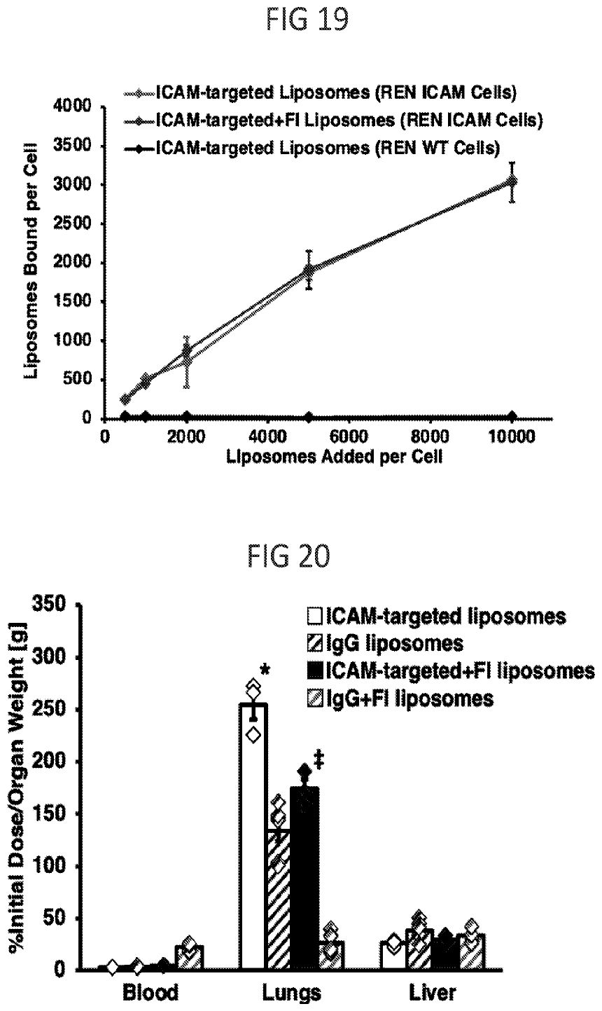

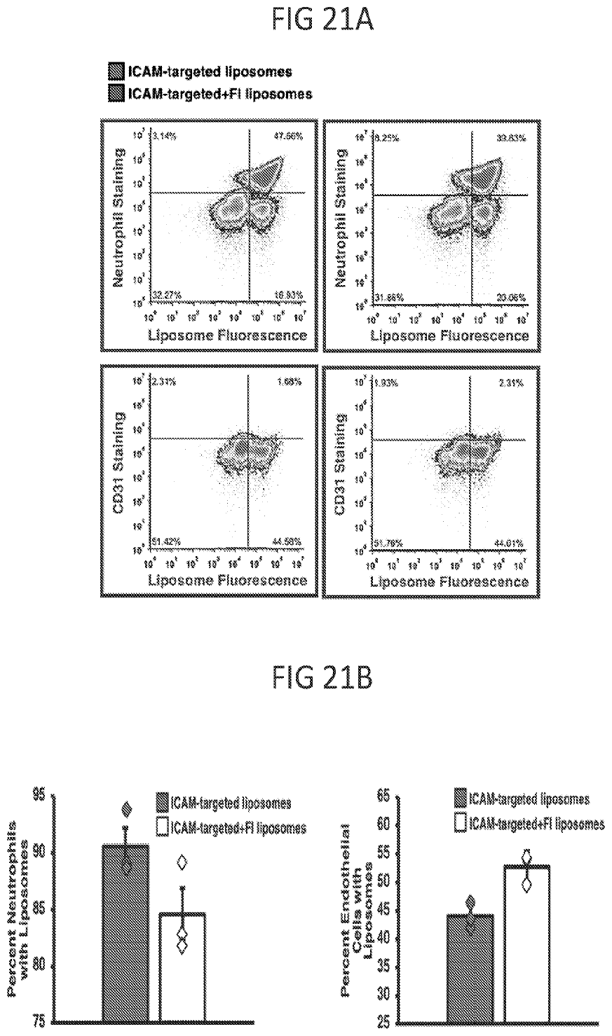

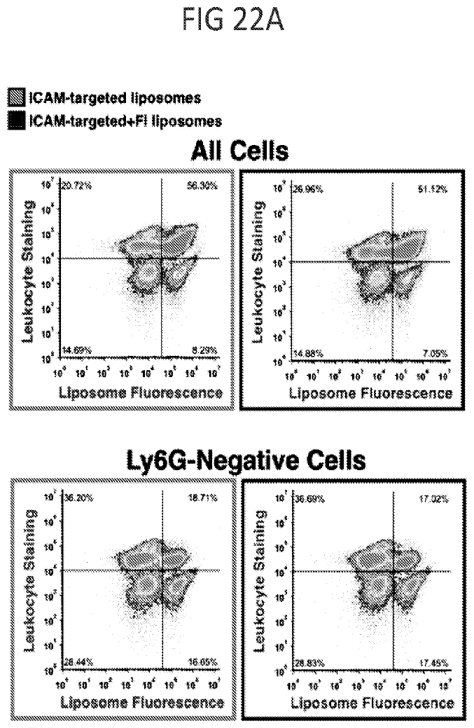

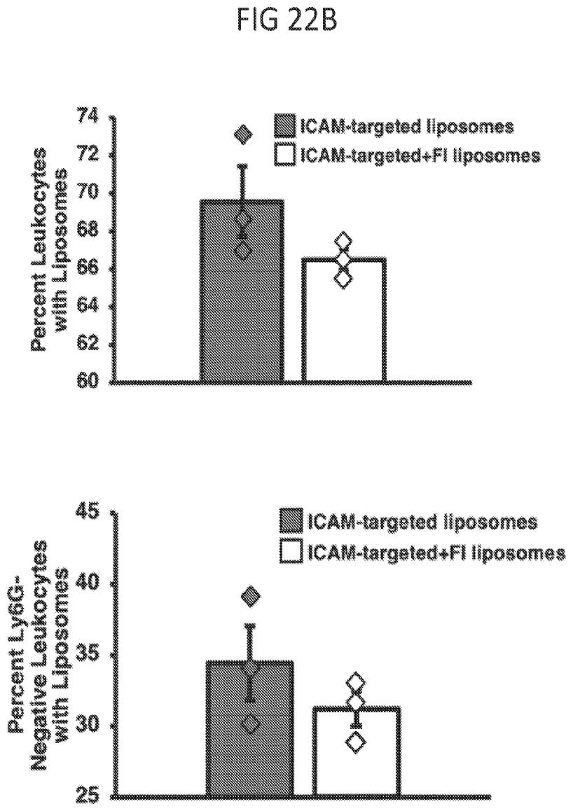

We next tested whether Factor I could similarly prevent cell-targeted nanoparticles from being taken up by local phagocytes. We first showed that, in vitro, anti-ICAM-liposomes specifically bind ICAM on the surface of target cells, and that Factor I conjugation does not affect the binding avidity (FIG. 19). We next injected anti-ICAM-liposomes into sepsis-model mice. As with IgG-liposomes, Factor I decreased ICAM-targeted nanoparticle uptake in the lungs (FIG. 3c; FIG. 20) and increased nanoparticle retention in the blood (FIG. 3c inset). Notably, while the absolute ICAM-targeted nanoparticle uptake in the lungs decreased for the anti-ICAM+Factor I liposomes, the ICAM-targeted:IgG lung uptake ratio increased to 6.5, compared to 1.9 for liposomes without Factor I. We next disaggregated the lungs into a single-cell suspension, and used flow cytometry to check if the above-observed decreased lung uptake was due to decreased uptake by phagocytes. Indeed, the mean nanoparticle fluorescence in neutrophils (the main local, intravascular leukocyte) decreased by 21.0% (FIG. 3D-1, left panel, FIG. 21A-B left panels). Similar analysis of all lung leukocytes showed that Factor I reduced uptake in the total population of leukocytes, though specific reduction of uptake in Ly6G-negative leukocytes (non-neutrophil leukocytes) was not significant (FIG. 22A-B). By contrast, the mean nanoparticle fluorescence increased 44.6% in endothelial cells, which are the intended target cell for anti-ICAM liposomes (FIG. 3D-2, FIG. 21A-B right panels). Thus, as hypothesized, Factor I surface conjugation prevented nanoparticle association with local phagocytes, and improved targeting to the cells of interest.

### Factor I Surface-Conjugation Prevents the Severe Anaphylactoid Responses Induced by Nanoparticles

All of the above investigations related to Factor I conjugation improving the beneficial aspects of nanoparticles by decreasing C3b-adduct formation, decreasing RES and local phagocyte uptake, and improving biodistribution and pharmacokinetics. However, complement is also known to cause severe side effects, so we next investigated Factor I's ability to prevent those. For decades, infusion of nanoparticles in animals and humans has been known to cause the anaphylactoid syndrome of CARPA^{[8,32,33]}. CARPA is characterized by rapid production of two complement pathway-produced anaphylatoxins: C3a, a weak anaphylatoxin; and C5a, among the strongest known anaphylatoxins. These products, and likely other complement products, lead to urticaria (rash) and rapid (within 10 minutes) systemic capillary leak, which leads to systemic hypotension, shock, and occasionally death. These issues have been partially addressed in clinical practice, with infusion of IV fluids and for some nanomedicines, such as patisiran (the first FDA-approved lipid nanoparticle to deliver nucleic acids), the requirement for pretreatment hours ahead with steroids. However, in patient populations which cannot tolerate even slight hypotensive stimuli, such as critically ill patients with sepsis or stroke, CARPA could make nanomedicine far too risky.

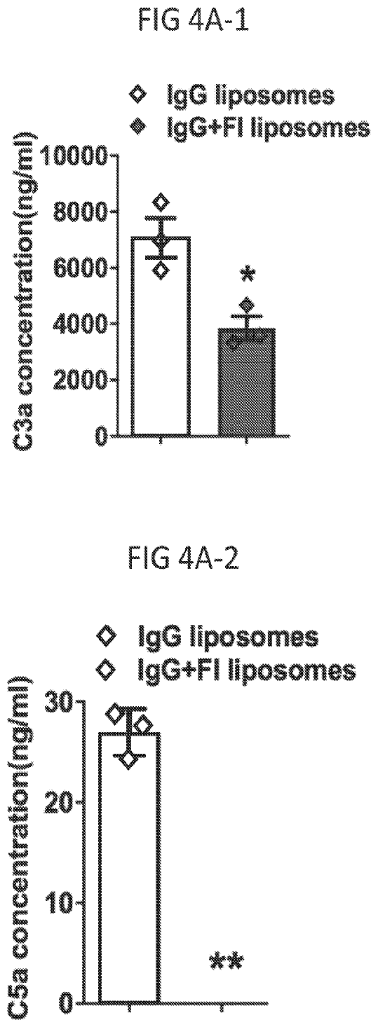

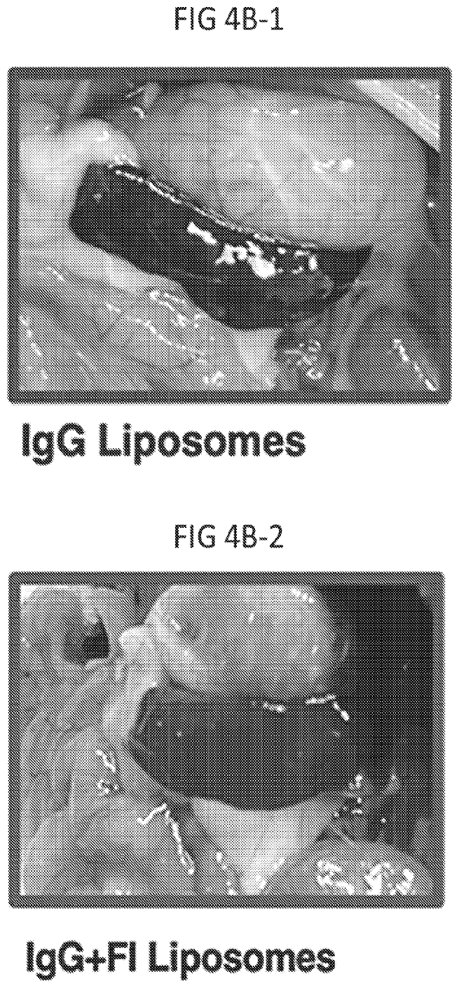

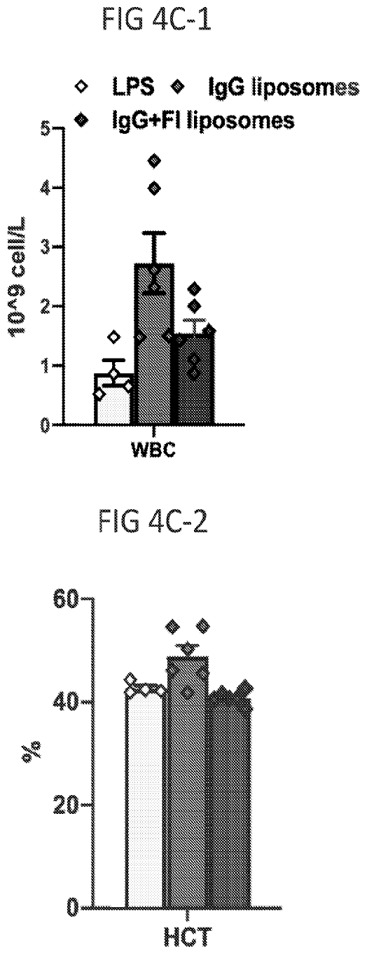

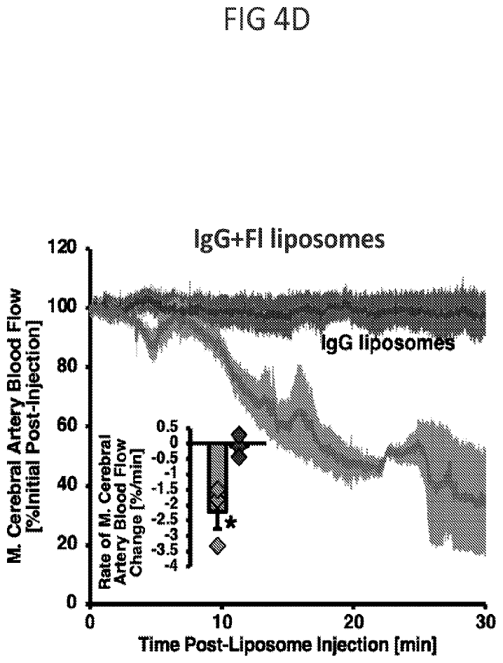

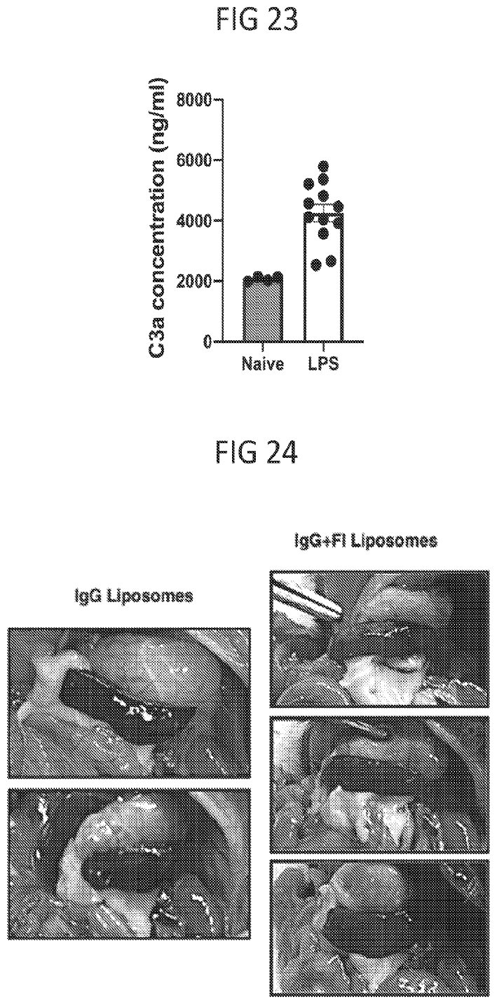

To study whether Factor I conjugation could prevent CARPA in such vulnerable, critically ill populations, we tested Factor I's ability to prevent CARPA and its attendant hypotension in the IV LPS mouse model of sepsis. Note that the complement cascade is already partially activated in IV LPS mice, as shown in FIG. 23, and prior studies^{[19,20]}. Into such IV LPS mice, we injected IgG-liposomes+/−Factor I, and sacrificed them 10 minutes later. Assaying C3a in the blood of these mice showed that Factor I decreased the C3a concentration by 45% (FIG. 4a, left panel). Even more impressively, Factor I decreased the most potent anaphylatoxin, C5a, to undetectable levels (FIG. 4a, right panel). Thus, in vivo, Factor I powerfully prevents production of complement's main anaphylatoxins.

We next examined whether Factor I can prevent the visceral organ hypoperfusion predicted to occur with CARPA. To do this, in the same mice in which we assayed C5a blood levels, we photographed the spleen, which can become dark during states of visceral organ hypoperfusion or hypoxia. As shown in FIG. 4b and FIG. 24, the spleens of mice receiving IgG-liposomes were very dark, almost black, whereas the spleens of mice receiving Factor I-conjugated IgG-liposomes were the normal light burgundy color of naive mice, suggesting Factor I protected from abdominal organ hypoperfusion.

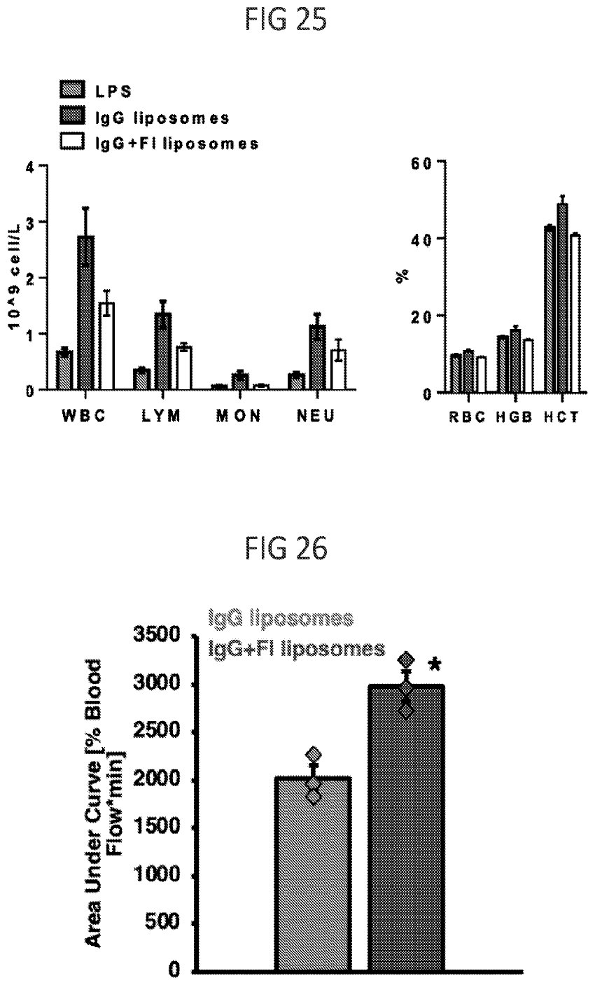

Next, we measured whether the transient leukocytosis previously described for CARPA is also observed in these IV LPS mice after injection of IgG-liposomes. FIG. 4c and FIG. 25 show that IgG-liposomes increase leukocyte concentration by 4.1-fold. Conjugation of Factor I reduced that nanoparticle-induced leukocytosis by 57.7%.

We next investigated if nanoparticles injected into IV-LPS mice also induce the massive capillary leak phenomenon of CARPA, previously shown in pigs. To measure capillary leak, we measured the hematocrit, which is a measure of the fraction of blood's volume that is occupied by red blood cells. In states of rapid capillary leak, the hematocrit increases (called “hemoconcentration”), as plasma leaks into the tissues. Indeed, we found that IgG-liposomes caused a 14.4% increase in the hematocrit 10 minutes after nanoparticle injection. Importantly, this hemoconcentration was completely abrogated by Factor I conjugation, suggesting that Factor I can indeed prevent capillary leak.

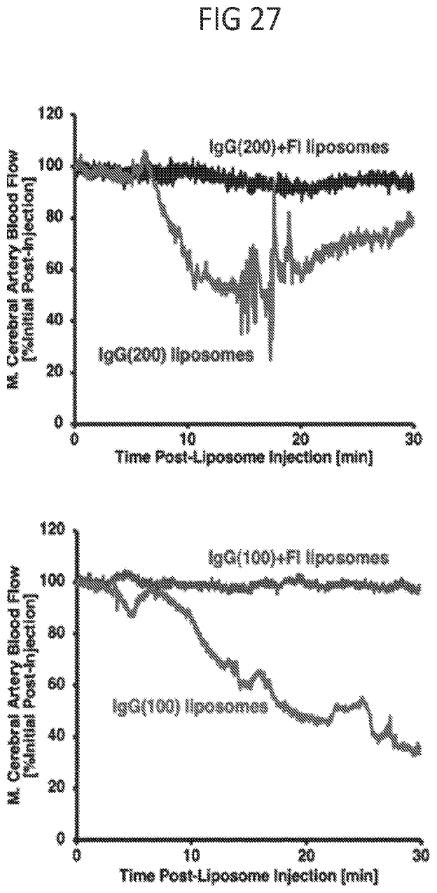

Finally, we finished by determining if Factor I can prevent hypoperfusion to the brain, the most sensitive organ during acute critical illnesses. Many groups have for decades pursued nanoparticle-based treatments for acute ischemic stroke. However, we hypothesized that CARPA from nanoparticles could induce cerebral hypoperfusion, which could make cerebral infarcts enlarge, as the at-risk, partially perfused “penumbra” region around a stroke's core is very sensitive to hypoperfusion. As hypothesized, IgG-liposomes decreased perfusion to the mouse brain (measured by Doppler ultrasound of the middle cerebral artery) by more than 60%, starting within 5 minutes after nanoparticle injection, and lasting at least 30 minutes (FIG. 4d; FIG. 26-27). As a comparator, in the transient middle cerebral artery occlusion (tMCAO) mouse model of ischemic stroke, a 70% reduction in middle cerebral artery blood flow is standardly considered sufficient to induce a large volume cerebral infarct (stroke)^{[34]}. Importantly, this cerebral hypoperfusion was completely prevented by Factor I conjugation. Analysis of middle cerebral blood flow by both average rate of blood flow reduction and area under curve showed significant improvement with Factor I (FIG. 4d, inset, FIG. 26). Conjugation to Factor I eliminated IgG liposome effects on middle cerebral artery blood flow for two densities of IgG on the liposomes, 200 or 100 IgG per liposome (FIG. 27). Thus, while nanoparticles could have outsized side effects in critically ill patients like those with strokes, Factor I may be able to prevent these and enable nanomedicine to move into the intensive care unit (ICU).

### DISCUSSION

Nanomedicine has faced several challenges on its quest to safely shuttle drugs to their desired location for a specified length of time. Among the greatest of these challenges has been created by the complement system, which has had a half-billion-year head start in designing a defensive system to prevent nano-scale particles, meaning microbes and later engineered nanoparticles, from going where they want in the body. Nanomaterials engineers have made significant strides against this defense, most notably with the introduction of hydrophilic polymer brushes, which have served as partial blockers of C3 reaching the nanoparticle surface. But clearly, more is needed, as most nanoparticles, with or without PEG or other brush polymers, end up in the RES organs rather than in their intended tissue^{[7]}.

However, nanoengineers have started to borrow from nature's armamentarium to fight complement. The first example was co-injecting complement inhibitors into the blood at the same time or right before nanoparticle injection^{[14,15]}. These elegant studies showed in vitro that regulators of complement activity (RCAs), such as Factor H, decreased complement activation when nanoparticles were added to serum. However, clinically, co-injection of long-acting complement inhibitors with nanoparticles comes with the significant danger of causing systemic complement inhibition, which is a powerful way to suppress the immune system. Such immunosuppression could be very dangerous, especially in hospitalized patients, who are already at greatly increased risk of acquiring new infections.

We conjugated RCAs directly onto the nanoparticle surface. Surface conjugation greatly increases the local concentration of the RCAs, and thus, at least theoretically, should require far lower numbers of RCA molecules to be introduced into patients, avoiding immunosuppression. We found that appending RCAs to the nanoparticle surface efficiently prevented C3 opsonization (FIG. 1), leading to numerous other benefits: decreased RES organ uptake and thus prolonged nanoparticle circulation in the blood (FIG. 2); less nanoparticle uptake by phagocytes, including local phagocytes in the target organ (FIG. 3); and decreased CARPA-associated side effects, such as systemic capillary leak, visceral hypoperfusion, and most importantly, cerebral hypoperfusion (FIG. 4). Thus, RCA surface conjugation, especially of Factor I, appears to be a way of overcoming many of the hurdles that have tripped up nanomedicine for years.

It is very unlikely that the amount of Factor I present on our nanoparticles would have an immunosuppressive effect. We are proposing the injection of very small masses of Factor I, as shown in the following dose calculations:

In humans, most IV liposomes (e.g., Doxil) are dosed at, maximally, 10 mg/kg^{[35]}. Converting that maximal dose to a 10 mg/kg mouse dose, for our average targeted liposomes in this study, that is 3×10¹¹ liposomes injected per mouse. In FIG. 4D, we showed that the optimal # of Factor I molecules per liposomes is 20. Thus, we are injecting 6×10¹² Factor I molecules into the 2 mL of blood that a mouse has, which, given the 88 kDa weight of Factor I, means we are injecting 0.4 ug of Factor I/mL of blood. By comparison, Factor I is present at 35 μg/mL in the blood^{[12]}). Thus, the Factor I technology proposed here will only increase the amount of Factor I in the blood by ˜1%, which is unlikely to cause immunosuppression.

It is surprising that Factor I worked as well as Factor H in our experiments. There was a previous report in which Factor H was physisorbed (non-covalent adsorption) onto silicon nanoparticles^{[17]}, and this adsorption did not reduce C3a activation in whole serum, and failed to reduce phagocyte uptake, suggesting that Factor H is very sensitive to the method of adsorption onto nanoparticles.

### Methods for Nanoparticle Preparation

Liposomes were prepared from 1,2-dipalmitoyl-sn-glycero-3-phosphocholine (DPPC), 1,2-distearoyl-sn-glycero-3-phosphoethanolamine-N-[azido(polyethylene gly-col)-2000 (azide PEG 2000 DSPE) and cholesterol (54:40:6 mol %) using the classical lipid thin film extrusion method as described previously^{[18]}. In brief, lipids were dissolved in chloroform, then added to together in the above molar ratios to a final lipid molarity of 20 nM, and the cholorform was evaporated by blowing nitrogen onto the liquid, resulting in a thin film at the bottom of a borosilicate tube. We hydrated the films by adding 500 μL of PBS, followed by bath sonication at 55 C. We then extruded the lipids through a 200-nanometer filter using an Avanti Polar Lipids syringe extruder. For fluorescent liposomes, an additional 1% of TopFluor PC was included in the formulation. Liposomes were then sized via dynamic light scattering (DLS; Malvern Zetasizer).

We next conjugated liposomes to specific proteins using the common “click chemistry” of strain-promoted alkyne-azide cycloaddition (SPAAC)g, as described previously.^{[18]} The method of modifying the specific proteins for SPAAC was is described in the section below, “Modification of Proteins.” The DBCO-modified proteins were covalently conjugated to the azide-functionalized liposomes by mixing the protein and liposomes in PBS and incubated at 4° C. overnight. The ratios of protein-to-liposome were determined by the desired # of proteins per liposome (see Results), assuming a conjugation efficiency of 60-90%. We then measured the # of actual proteins per liposome via Sepharose column (see below), and adjusted reaction ratios to achieve our goal # of proteins per liposome. For further details on conjugation of proteins to liposomes, see the section below,

### 4.4. Characterization of Protein Conjugation to Liposomes

Immunoliposomes were purified from residual antibodies using a 20 mL Sepharose 4B-Cl column (GE Healthcare, Pittsburgh, PA). DLS measurement of hydrodynamic particle size and polydispersity index using a Zetasizer Nano ZSP (Malvern Panalytical, Malvern UK). The resulting liposome concentration in number per ml was measured using NanoSight NS300 (NanoSight, Salisbury, United Kingdom) using a 4×10⁴ dilution into high purity DI water.

### Methods for the Modification of Proteins

Proteins were conjugated to azide functionalized liposomes via copper-free click chemistry^{[18]}, antibodies (whole molecule IgG, Thermofisher; anti-ICAM mAb YN1/1.7.4 grown from hybridoma per ATCC) and complement factors (Factor H and I, Complement Technology, Inc. Tyler, TX) were modified using DBCO-PEG4-NHS ester (Jena Bioscience, Jena, Germany) according to the manufacturer's protocol. Briefly, antibodies were mixed with NHS ester in DMSO at 1:20 molar ratio, and Factor H or Factor I at 1:5 molar ratio. After reaction for 30 min at room temperature, modified antibodies were purified from residual DBCO reagent and free NHS ester using Amicon Ultracel-50 kDA membrane filter (Millipore, Burlington, MA) to remove unreacted NHS ester PEG4 DBCO. The efficiency of DBCO-IgG reaction was determined optically, with absorbance at 280 nm indicating IgG concentration and absorbance at 309 nm indicating DBCO concentration. Spectral overlap of DBCO and IgG absorbance was noted by correcting absorbance at 280 nm. Molar protein concentration was determined using Beer's Law calculation. The number of DBCO per IgG was determined as the ratio. Secondary fluorescent labeling of proteins using Alexafluor 488 NHS ester (Thermofisher) followed manufacturer's instructions.

### Methods for the Radiolabeling of Antibodies

Antibodies were radioiodinated with [¹²⁵I]Na (Perkin Elmer, Waltham, MA) using Pierce lodgen radiolabeling reagent and purified using Zeba desalting spin columns (ThermoFisher Scientific). Radiochemical purity was assessed via TLC using a mobile phase of 75% methanol: 25% NH₄ acetate, and confirmed >90% in all cases. To radiolabel immunoliposomes, 2% radiolabeled untargeted (random IgG) antibodies were added for conjugation.

### Methods for the Characterization of Protein Conjugation to Liposomes

Proteins, including Factor H or I, IgG or mAb, modified with DBCO were combined in designated amounts and incubated with azide functionalized liposomes. Combining the measured Nanosight NTA liposomes concentration values in #/ml, and spectrophotometric protein concentrations, we combined mixtures of the DBCO functionalized proteins (including <10% by volume relative to total protein of labeled tracer protein), with azide liposomes for simultaneous conjugation at 4 C overnight. Calculations include an efficiency estimate from 60-90% of protein binding. For example, if IgG DBCO is estimated to have 90% binding efficiency and the target coating density is 100 IgG/liposome then we will calculate an addition of 110 IgG per particle. After conjugation incubation, the reaction mixtures were characterized and purified by size exclusion chromatography (SEC) using Sepharose 4B-CL (Sigma Aldrich) as previously described^{[18]}. Protein conjugation was quantified by tracing ligand fluorescence or radioactivity (each fraction read on a plate reader or a gamma counter). Efficiency of conjugation reaction is quantitatively defined as the ratio of the area under the curve of the ligand signal in the liposome peak (7-9 mL) over the sum of that peak combined with the free protein peak. FIG. 6 shows chromatography data of a titration of Factor I coating on liposomes, and 2 IgG coating densities, all indicating characteristic elution peaks. Fractions containing protein bearing liposomes are collected and re-concentrated using Amicon filtration devices (Millipore), then measured again for size and concentration using DLS and NTA as described above.

### Methods for C3a and C5a ELISA

ELISA testing was conducted to measure the activated C3a and C5a levels in vitro and in vivo, per manufacturer protocol. Briefly, for in vitro measurement, 20 μL of fresh serum was incubated with 20 μL of immunoliposomes (2×10¹² liposomes/mL, conjugated with designated DBCO modified proteins) for 15 minutes, EDTA was added to a final concentration of 20 mM, to inhibit further complement activation. For in vivo measurement, plasma was collected from mouse inferior vena cava with EDTA coated syringes, and then chelated with 20 mM EDTA and the pan-complement inhibitor Futhan (0.05 mg/ml, BD Pharmingen) to inhibit further complement activation. Serum/plasma C3a and C5a levels were measured by using sandwich ELISA kits from BD Biosciences Company.

### Methods for Nanoparticle Tracking Analysis for C3 Adhesion to Liposomes

To prepare fluorophore-labeled C3, human complement protein C3 (Complement Technology) was incubated with NHS ester Alexa Fluor 488 (ThermoFisher) at 1:5 mol:mol ratio in PBS with 0.1 M NaHCO₃ on ice for two hours. Afterwards, excess fluorophore was removed from C3 by 3-fold passage against molecular weight cutoff 10 kDa centrifugal filter (Amicon) with PBS washing between passages. After C3 recovery from the centrifugal filter, spectrophotometer measurement of optical density at 280 nm determined fluorescent C3 concentration and optical density measurement at 488 nm determined the number of fluorophores per C3.

Immediately before experiments, liposome concentrations were determined by nanoparticle tracking analysis (Nanosight, Malvern). In a total reaction volume of 40 μL, 4×10¹⁰ liposomes were combined with 20 μL mouse serum and fluorescent C3 was doped into the solution at a final concentration of 0.3 mg/mL. Fluorescent C3, serum, and liposomes were incubated in the dark at room temperature for 20 minutes. Fluorescent C3 was also added to serum solutions at identical concentration, without liposomes, verifying that the fluorescent C3 did not adhere to endogenous serum components at detectable concentrations. The C3-serum-liposomes reactions were terminated by 1:250 dilution in PBS and the diluted suspensions were used for nanoparticle tracking analysis. Nanoparticle tracking analysis was conducted with a 488 nm excitation laser and a 500 nm long pass filter to image and track Alexa Fluor 488 signal from fluorescent C3 on nanoparticles. Automated analysis of fluorescence nanoparticle tracking data in Malvern Nanosight software used a uniform detection threshold of 5 for all samples. The same samples were immediately analyzed with an open filter to assess light scattering species, rather than just fluorescent-tagged species, therefore imaging and tracking all serum components and unlabeled liposomes in the sample and verifying that the fluorescent population was distinct from the total population of serum components in its size distribution and concentration. Scattering-based nanoparticle tracking data was analyzed in Malvern Nanosight software with a detection threshold of 12. For both fluorescence data and scattering data, five technical replicates were obtained for each sample and an average of those replicates was taken as representative of the size-concentration profile for each sample.

### Methods for Animal Studies: Pharmacokinetics and Biodistribution of Immunoliposomes in Naive and Inflamed Mice

Immunoliposomes (3 mg/kg, ˜2×10¹² liposomes/mL) were intravenously injected in naive or lipopolysaccharide (LPS) treated groups. For LPS groups, mice were anesthetized with 3% isoflurane, LPS from E. coli strain B4 (Sigma) was administered at 2 mg/kg in 100 L PBS 5 hours prior to liposome injection. After five hours, mice were anesthetized with ketamine-xylazine (10 mg/kg ketamine, 100 mg/kg xylazine, via intramuscular administration) and were injected intravascularly with 3 mg/kg immunoliposomes conjugated with designated DBCO modified proteins (IgG, anti ICAM YN1, Factor I). The animals were euthanized at designated times after injections (30 minutes after nanoparticle injection, unless otherwise stated), and the organs of interest were harvested, rinsed with saline, blotted dry, and weighed. Blood samples (˜200 ul) were spun down at 500 rcf in a microcentrifuge tube with RBCs separated from plasma. Biodistribution quantification was determined by measuring the radioactivity in the blood and other tissues using a Wallac 2470 Wizard gamma counter (PerkinElmer Life and Analytical Sciences-Wallac Oy, Turku, Finland). The gamma data of the ¹²⁵I measurements and organ weights were used to calculate the tissue biodistribution injected dose per gram of tissue. The total injected dose was measured prior to injections, corrected for tube and syringe residuals, and verified to be ≥75% of the sum of the individual measures.

Blood pharmacokinetic data was analyzed via standard noncompartmental analysis (NCA) in order to derive the area under the concentration vs. time curve (AUC), which was calculated using the linear trapezoidal rule. This can be done using MATLAB's Simbiology NCA system.

### Methods for In Vitro Neutrophil Uptake of Liposomes

Neutrophils were purified from ˜8-week-old C57BL/6 mouse femur bone marrow. Femurs were harvested after euthanasia, their ends were cut off, and the marrow was extracted by flushing media through the cut end. The marrow cells were then subjected to magnetic bead pull down of non-neutrophils using RoboSep Mouse Neutrophil Enrichment Kit (StemCell Technologies), exactly according to manufacturer instructions. The neutrophils were placed into 500 uL media at a concentration of approximately 2×10⁶ cells/mL.

Within 1 hour of neutrophil isolation, 1×10⁶ neutrophils were rotated with 5×10⁹ liposomes in 20 μL PBS for 15 minutes at 37° C. Alternatively, serum-treated liposomes were prepared by incubating 5×10⁹ fluorescent liposomes in 10 μL PBS with 10 μL of mouse serum for one hour at 37° C. prior to addition to 1×10⁶ neutrophils. The mouse serum was prepared by drawing blood from wild-type mice, allowing the blood to coagulate in a 1.5 mL Eppendorf centrifuge tube for 30 min at room temperature, and then centrifuging at 1500 g×10 minutes at 4 C. For flow cytometry (BD Accuri C6), neutrophils were washed and stained with PerCP/Cy5.5 Ly6G antibodies (BD Biosciences, 1:100 dilution) and non-neutrophils were excluded from analysis via Ly6G staining (see FIG. 18A-D for gating). Liposome fluorescence in neutrophils was quantified by mean fluorescence intensity, gated on Ly6G-positive populations.

### Flow Cytometry Analysis of ICAM-Targeted Liposome Distribution Among Cell Types in the Lungs

Mice were injected with LPS (2 mg/kg, IV) 5 hours prior to IV-injection of fluorescent (1% of TopFluor PC) anti-ICAM-liposomes+/−surface-conjugated Factor I. Mice were anesthetized with ketamine/xylazine (10 mg/kg ketamine, 100 mg/kg xylazine, intramuscular administration) in order to place a tracheal catheter secured by suture. Thirty minutes after liposome administration, mice were sacrificed by terminal exsanguination via the vena cava and lungs were perfused by right ventricle injection of ˜10 mL of cold PBS. The lungs were then infused via the tracheal catheter with 1 mL of a digestive enzyme solution consisting of 5 U/mL dispase, 2.5 mg/mL collagenase type I, and 1 mg/mL of DNAse I in cold PBS. Immediately after infusion, the trachea was sutured shut while removing the tracheal catheter. The lungs with intact trachea were removed via thoracotomy and kept on ice prior to manual disaggregation by vigorous chopping with scissors and razors.

Disaggregated lung was aspirated in an additional 2 mL of digestive enzyme solution and incubated at 37° C. for 45 minutes, with vortexing every 10 minutes. After addition of 1 mL of fetal calf serum, tissue suspensions were strained through 100 μm filters and centrifuged at 500×g for 5 minutes. After removal of supernatant, the pelleted material was resuspended in 10 mL of cold ACK lysing buffer. The resulting suspensions were strained through a 40 μm filter and incubated for 10 minutes on ice. The suspensions were centrifuged at 500×g for 5 minutes and the resulting pellets were rinsed in 10 mL of FACS buffer (2% fetal calf serum and 1 mM EDTA in PBS). After centrifugation at 500×g for 5 minutes, the rinsed cell pellets were resuspended in 2% PFA in 1 mL FACS buffer for 10 minutes incubation at room temperature in the dark. The fixed cell suspensions were centrifuged at 500×g for 5 minutes and resuspended in 1 mL of FACS buffer.

To stain fixed cells, 100 μL aliquots of cell suspensions were pelleted 500×g for 5 minutes, then resuspended in labeled antibody diluted in FACS buffer (1:150 dilution for Alexa Fluor 647 anti-Ly6G or APC-anti-CD31 and 1:500 dilution for PerCP/Cy5.5 anti-CD45). Samples were incubated with staining antibodies for 20 minutes at room temperature in the dark, diluted with 1 mL of FACS buffer, and pelleted at 500×g for 5 minutes. Stained pellets were resuspended in 200 μL of FACS buffer immediately prior to flow cytometry analysis (BD Accuri). Data was gated on FSC vs. SSC and FSC (height) vs. FSC (area) to exclude debris and doublets. Controls with no stain, obtained from IV-LPS-injured mice not receiving fluorescent nanoparticles, established gates for negative/positive staining with TopFluor PC liposomes, Alexa Fluor 647- or APC-labeled antibodies, or PerCP/Cy5.5-labeled antibodies. Single stain controls allowed automatic generation of compensation matrices in FCS Express software during final analysis of the data. Association of liposomes with cell types was identified by coincidence of green fluorescent signal with anti-CD45, anti-Ly6G, or anti-CD31 signal.

Method for Brain Doppler Blood Flow Measurements of Inflamed Mice Treated with Liposomes

Mice were injected with LPS (2 mg/kg, IV) 5 hours prior to measuring the blood flow, using a moorVMS-LSD laser doppler perfusion system. Anesthetized mice (2% isoflurane) were placed on a heating pad with a rectal probe to keep the temperature at 37+/−0.5 C. An incision was made between the ear and the eye, the masseter muscle was separated from the skull to find the middle cerebral artery (MCA). The doppler probe was fixed over the MCA and the blood flow monitored for 2 minutes prior to the injection of the liposomes (3 mg/kg). Blood flow was followed 30 minutes after the injection of liposomes. Isoflurane was reduced to 1% if the blood flow was reduced by a 50% of the baseline (which only happened in the mice that received liposomes without Factor I) to prevent animal death. Finally, mice were ethically euthanized and the blood collected for complete blood count (CBC).

### Methods for Animal Protocols

All animal studies were carried out in strict accordance with Guide for the Care and Use of Laboratory Animals as adopted by National Institute of Health and approved by University of Pennsylvania Institutional Animal Care and Use Committee (IACUC). Male C57BL/6J mice, 6-8 weeks old, were purchased from Jackson Laboratories. Mice were maintained at 22-26° C. and on a 12/12 hour dark/light cycle with food and water ad libitum.

### Methods for Statistics and Software

All statistics were analyzed with GraphPad Prism, with the tests used indicated in the figure legends.

### Example 2: Aptamer-Nanoparticles

We previously showed that conjugating the protein Factor H (FH) to the surface of nanoparticles prevents activation of complement (C3, C5, etc) on the surface of the nanoparticle. Because recombinant proteins such as FH are difficult to manufacture at large scales, we sought to make a nanoparticle that would bind the Factor H that is naturally in human blood.

To make an FH-binding nanoparticle, we first found in the literature a FH-binding aptamer. An aptamer is a DNA sequence that folds and then binds to a target. We conjugated the FH-binding aptamer to nanoparticles, and showed that the nanoparticles bind Factor H.

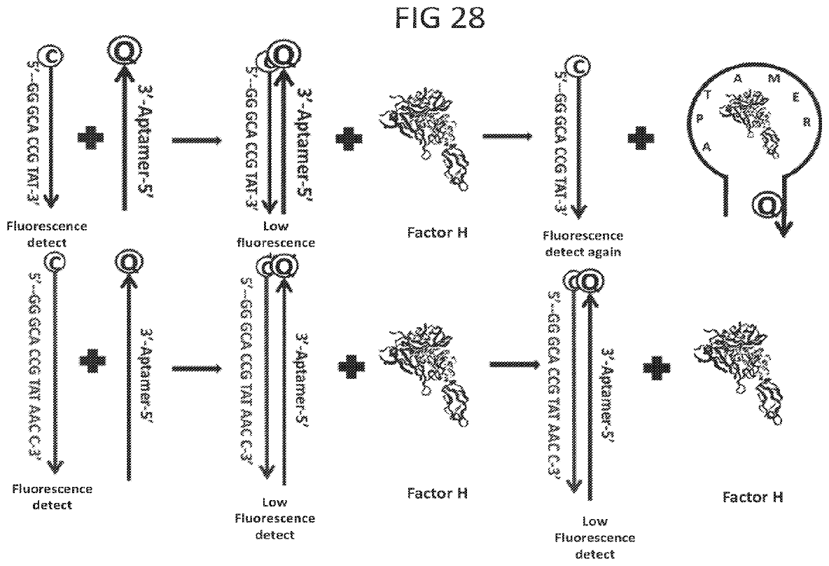

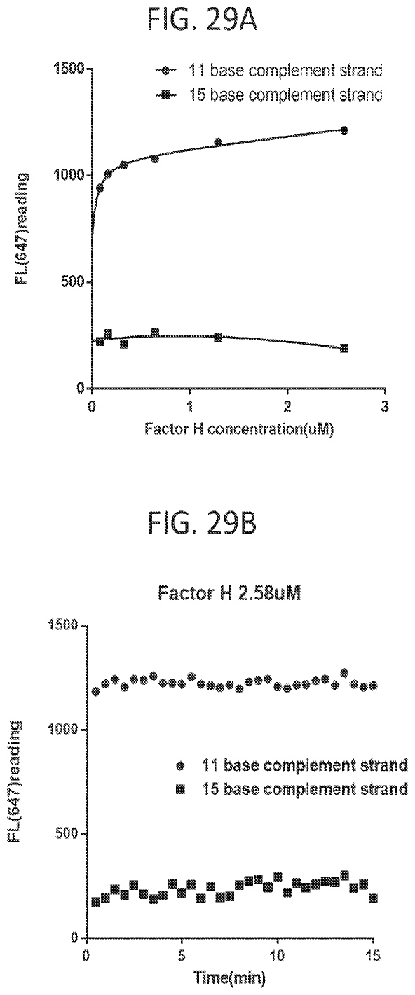

The assay which we used to show Factor H binding to nanoparticles is shown in FIGS. 28-29. We synthesized 3 DNA sequences: 1) the aptamer with a fluorophore on it so it will fluoresce; 2) an 11-base-pair (11-bp) “complementary” strand of DNA that binds to the aptamer via a complementary sequence (e.g., AGT binds to TCA). This 11-bp sequence has a “quencher” molecule on its end that will quench (reduce) the fluorescence of the aptamer, but only if the aptamer is bound to the 11-bp strand. Thus, when the aptamer and 11-bp strand are mixed, the fluorescence is reduced. 3) a 15-bp complementary strand.

1. Factor H aptamer:
(SEQ ID NO: 5)
5′-GGT CTC GGG CAC GGG TCA GGC GGT TAT ACG GTG
CCC-BHQ-3′
2. Complement strand 11 base:
(SEQ ID NO: 6)
5′-Cy5-GG GCA CCG TAT-3′
3. Complement strand 15 base:
(SEQ ID NO: 7)
5′-Cy5-GG GCA CCG TAT AAC C-3

We first mix the complementary strand (11- or 15-bp) with the aptamer, which reduces fluorescence compared to the aptamer alone. We then add Factor H. If Factor H binds to the aptamer, the complementary strand, along with its quencher, will be displaced, and thus fluorescence will increase.

We showed that adding Factor H does indeed displace the complementary 11-bp strand, even at low amounts of Factor H, showing this is strong binding. Thus, our Factor H aptamer does indeed work to bind Factor H.

Nanoparticles conjugated to a Factor H-binding aptamer efficiently bind Factor H (FIG. 29A). The aptamers were first exposed to complementary strands that quenched a fluorophore attached to the aptamer, but addition of Factor H displaces the quencher at very low Factor H concentrations indicating strong binding. This displacement works for as much as 11-base complementary strands (less for 15-base), which indicates strong Factor H binding.

Titration of Factor H concentrations added to double strand complex. Fluorescence increase is observed due to the release of BHQ-labeled complement strand by adding factor H to 11-nt aptamer-complement duplex. While no significant fluorescence increase is observed for adding factor H to 15-nt aptamer-complement duplex. The reaction is completed in a few mins.

Each and every patent, patent application, and publication is incorporated herein by reference. U.S. Provisional Patent Application No. 63/286,938, filed Dec. 7, 2021 is incorporated by reference in its entirety. While the invention has been described with reference to particular embodiments, it will be appreciated that modifications can be made without departing from the spirit of the invention. Such modifications are intended to fall within the scope of the appended claims.

1. A composition comprising:
a) a therapeutic molecule, diagnostic molecule, molecular complex, or nanoparticle having a complement inhibitory protein attached thereto; or
b) a therapeutic molecule, diagnostic molecule, molecular complex, or nanoparticle having a peptide or aptamer that binds a complement inhibitory protein attached thereto.

2. (canceled)

3. The composition according to claim 1,
a) wherein the nanoparticle is a lipid nanoparticle or liposome;
b) wherein the nanoparticle is a protein-coated nanoparticle or polymeric nanoparticle;
c) further comprising a targeting moiety or moieties; and/or
d) wherein the complement inhibitory protein is selected from those in Table 1, or a functional variant or functional fragment thereof;
e) wherein the aptamer has a sequence of SEQ ID NO: 5, or a sequence having 1, 2, 3, 4, 5, 6, 7, 8, 9, or 10 substitutions as compared to SEQ ID NO: 5; and/or
f) wherein the therapeutic molecule, molecular complex, or nanoparticle is attached to the complement inhibitory protein by a flexible linker.

4-9. (canceled)

10. The composition according to claim 9, wherein
a) the linker comprises polyethylene glycol; and/or
b) the linker is from 500 to 2500 D.

11. (canceled)

12. The composition according to claim 1, wherein the complement inhibitory protein is a variant that includes unnatural amino acids and/or is truncated.

13. (canceled)

14. The composition according to claim 1, wherein the therapeutic molecule, molecular complex, or nanoparticle comprises a radionuclide.

15. (canceled)

16. The composition according to claim 1, comprising least or about 10 to about 30 complement inhibitory protein molecules per nanoparticle.

17. The composition according to claim 1, comprising at least or about 10 to about 30 peptides or aptamers that bind a complement inhibitory protein per liposome or nanoparticle.

18. The composition according to claim 1, wherein the liposome or nanoparticle comprises a therapeutic or diagnostic molecule or multimolecular complex encapsulated therein.

19. (canceled)

20. A medical device resistant to opsonization comprising:
a) the medical device having a complement inhibitory protein attached thereto; or
b) the medical device having a peptide or aptamer that binds a complement inhibitory protein attached thereto.

21. (canceled)

22. The medical device according to claim 20, wherein
a) the medical device comprises medical tubing, dialysis tubing, or a membrane;
b) the medical device comprises an implant, catheter, tubing for medical devices that contacts blood (e.g., cardiopulmonary bypass machines and hemodialysis/hemofiltration machines), functional components of medical devices that contact human blood (e.g., oxygenators used in extracorporeal membrane oxygenation; dialysis and ultrafiltration membranes used in renal replacement therapy);
c) the complement inhibitory protein is selected from those of Table 1
d) the aptamer has a sequence of SEQ ID NO: 5, or a sequence having 1, 2, 3, 4, 5, 6, 7, 8, 9, or 10 substitutions as compared to SEQ ID NO: 5; and/or
e) the medical device is attached to the complement inhibitory protein by a linker.

23-27. (canceled)

28. The medical device according to claim 22, wherein:
a) the linker comprises polyethylene glycol; and/or
b) the linker is from 500 to 2500 Da.

29. (canceled)

30. The medical device according to claim 20, wherein the complement inhibitory protein is a variant that includes unnatural amino acids and/or is truncated.

31-32. (canceled)

33. The medical device according to claim 20, wherein the device encapsulates or releases a therapeutic or diagnostic molecule or multimolecular complex.

34. The medical device according to claim 33, wherein the therapeutic or diagnostic molecule is rapamycin, paclitaxel, zotarolimus, or everolimus.

35. A method comprising attaching a complement inhibitory protein, or an aptamer or peptide that binds a complement inhibitory protein, to a therapeutic molecule, molecular complex, liposome, or nanoparticle to generate a therapeutic composition capable of prolonged circulation time when administered to a subject.

36. A method of preventing or reducing the severity of complement-activation-related pseudoallergy (CARPA) in a subject in need of therapeutic treatment, the method comprising administering to the subject a composition comprising a therapeutic molecule, molecular complex, liposome, or nanoparticle having a complement inhibitory protein or an aptamer or peptide that binds a complement inhibitory protein, attached thereto, wherein opsonization of the composition is prevented or reduced, thereby preventing or reducing the severity of CARPA in the subject.

37. A method of treating in a subject in need of therapeutic treatment, the method comprising administering to the subject a composition comprising a therapeutic molecule, molecular complex, liposome, or nanoparticle having a complement inhibitory protein or an aptamer or peptide that binds a complement inhibitory protein, attached thereto, wherein the circulation time of the therapeutic molecule, molecular complex, liposome, or nanoparticle is increased as compared to the same therapeutic molecule, molecular complex, liposome, or nanoparticle without the complement inhibitory protein attached thereto.

38. The method according to claim 35, wherein:
a) the composition is the composition of claim 1;
b) the therapeutic molecule, molecular complex, liposome, or nanoparticle comprises: mRNA, miRNA, siRNA, or DNA; protein therapeutics, including antibodies, enzymes, decoy receptors; peptides; small molecule drugs; with any of these drugs functioning as anti-hypertensives, vasopressors/inotropes, pro- or anti-coagulants, anti-inflammatories, antimicrobials, immunomodulators, nutrients, anti-cancer agents (chemotherapeutics), growth factors, or inhibitors of enzymes;
c) the nanoparticle is a lipid nanoparticle;
d) the nanoparticle is a protein-coated nanoparticle;
e) the complement inhibitory protein is selected from those of Table 1; and/or
f) the complement inhibitory protein is a variant that includes unnatural amino acids and/or is truncated; and/or
g) a targeting moiety is conjugated to the therapeutic composition.

39-46. (canceled)

47. The method according to claim 38, wherein;
a) the linker comprises polyethylene glycol;
b) the linker is at least 500 Da; and/or
c) the linker is from 500 to 2500 Da.

48-51. (canceled)

52. The method according to claim 35,
a) wherein the therapeutic molecule, molecular complex, nanoparticle comprises a radionuclide;
b) comprising at least 10 complement inhibitory protein molecules per liposome or nanoparticle;
c) comprising at least 20 complement inhibitory protein molecules per liposome or nanoparticle; and/or
d) wherein the liposome or nanoparticle comprises a therapeutic or diagnostic molecule encapsulated therein.

53-60. (canceled)


SEQUENCE LISTING (15 sequences)

SEQ ID NO: 1 - DNA, 1752 bp, Homo sapiens
  source 1..1752: mol_type = genomic DNA
atgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataa

SEQ ID NO: 2 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 3 - DNA, 3696 bp, Homo sapiens
  source 1..3696: mol_type = genomic DNA
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 4 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 5 - DNA, 36 bp, synthetic construct
  source 1..36: mol_type = other DNA
ggtctcgggcacgggtcaggcggttatacggtgccc

SEQ ID NO: 6 - DNA, 11 bp, synthetic construct
  source 1..11: mol_type = other DNA
gggcaccgtat

SEQ ID NO: 7 - DNA, 15 bp, synthetic construct
  source 1..15: mol_type = other DNA
gggcaccgtataacc

SEQ ID NO: 8 - RNA, 111 nt, synthetic construct
  source 1..111: mol_type = other RNA
ggagctcagccttcactgcggtctatatcgaggggggacgagaggggatgtatagacccaggtgaggccttggggacaatttgaatcgggcgtggcaccacggtcggatcc

SEQ ID NO: 9 - RNA, 109 nt, synthetic construct
  source 1..109: mol_type = other RNA
ggagctcagccttcactgctagtgaccgatcaggaggggagaaggaggacaccgtgtcaactacgccgtgtgaaggattgcagctgaggatggcaccacggtcggatcc

SEQ ID NO: 10 - RNA, 36 nt, synthetic construct
  source 1..36: mol_type = other RNA
ccgaccttagtcaatcacttcgttcgatgaatagca

SEQ ID NO: 11 - RNA, 35 nt, synthetic construct
  source 1..35: mol_type = other RNA
taaagcgtcgggccgtactgtcaatacatacaatg

SEQ ID NO: 12 - RNA, 36 nt, synthetic construct
  source 1..36: mol_type = other RNA
ggtttcaatgtcccggtactgtcatcacatacaatg

SEQ ID NO: 13 - RNA, 36 nt, synthetic construct
  source 1..36: mol_type = other RNA
gcttatcgtgtcttggtactgtcagcacatactatg

SEQ ID NO: 14 - DNA, 63 bp, synthetic construct
  source 1..63: mol_type = other DNA
aataaacgctcaactcaagtggcgtgcggcaggttggtgtgacggctgggagggttcgacatg

SEQ ID NO: 15 - DNA, 80 bp, synthetic construct
  source 1..80: mol_type = other DNA
ctcctctgactgtaaccacgttaggcgagaacatgtcagtacgtcgacgttctacttgctgcataggtagtccagaagcc